(19) United States
(12) Patent Application Publication
(10) Pub. No.: US 2025/0326733 A1
(43) Pub. Date: Oct. 23, 2025

(54) A NEW ROUTE FOR SYNTHESIZING AXIALLY CHIRAL CANNABINOIDS FROM COUMARINS

(71) Applicant: University of Florida Research Foundation, Inc., Gainesville, FL (US)

(72) Inventors: Alexander James Grenning, Gainesville, FL (US); Sara E. Kearney, Gainesville, FL (US)

(21) Appl. No.: 18/707,666

(22) PCT Filed: Dec. 9, 2022
(86) PCT No.: PCT/US2022/081229

§ 371 Date: May 6, 2024

Related U.S. Application Data
(60) Provisional application No. 63/265,231, filed on Dec. 10, 2021.
(60) Provisional application No. 63/267,513, filed on Feb. 3, 2022.

(51) Int. Cl. C07D 311/80; B01J 27/12; B01J 31/02; B01J 31/22; B01J 31/24; B01J 31/26

(52) U.S. Cl. CPC C07D 311/80; B01J 27/12; B01J 31/0224; B01J 31/2295; B01J 31/2409; B01J 31/26; B01J 2231/324; B01J 2531/004; B01J 2531/0205; B01J 2531/822

(57) ABSTRACT

In one aspect, the disclosure relates to axially chiral cannabinoid analogs and methods of making the same. In one aspect, several tetracyclic scaffolds can be prepared from O-propargyl vinyl coumarins in good yields. In a further aspect, these tetracyclic scaffolds can be treated with a reductant to form the axially chiral cannabinoid analogs. In another aspect, the axially chiral cannabinoid analogs are shelf stable and maintain a three-dimensional structure during storage, enabling superior recognition of biological targets such as cannabinoid receptors. Also disclosed herein are prochiral cannabinoid analogs that can be synthesized from axially chiral cannabinoid analogs.

### CROSS-REFERENCE TO RELATED APPLICATIONS

This application claims the benefit of U.S. Provisional Application No. 63/267,513, filed on Feb. 3, 2022, and U.S. Provisional Application No. 63/265,231, filed on Dec. 10, 2021. Each application is incorporated herein by reference in its entirety.

### STATEMENT REGARDING FEDERALLY SPONSORED RESEARCH OR DEVELOPMENT

This invention was made with government support under grant number R35 GM137893-01 awarded by the National Institute of General Medical Sciences of the National Institutes of Health. The government has certain rights in the invention.

### BACKGROUND

Phytocannabinoids and their synthetic analogs are prime candidates for pharmaceutical innovation in the quest for alternatives to highly addictive opioid analgesics, though they have yet to achieve FDA approval for this formidable goal. More generally, cannabinoid-based chemical probes and leads are essential for continued exploration of the endocannabinoid system, a complex neuro- and immunomodulating network implicated in a variety of neurodegenerative diseases as well as inflammation, metabolic disorders, and cancer. Most phytocannabinoid research to date has focused on the natural trans-tetrahydrocannabinol (trans-THC) and cannabidiol (CBD) frameworks, which has led to several approved medications (FIGS. 1A-1B). For example, (−)-trans-Δ⁹-THC is FDA approved (dronabinol) for the treatment of HIV/AIDS-induced anorexia as well as chemotherapy-induced nausea and vomiting. The approval of CBD (EPIDOLEX®, available from Jazz Pharmaceuticals, Cambridge, UK) to treat refractory childhood seizures marked the first time a Cannabis-based product was approved by the FDA.

Synthetic cannabinoids inspired by THC have emerged due to well-established synthetic protocols dating back to the 1940s via a renaissance of research in the late 20^{th} century. Additionally, there are numerous inspiring routes to CBD and minor cannabinoids. In this regard, nabilone is approved to treat chemotherapy-induced nausea and vomiting, and ajulemic acid has reached various clinical trial phases as a treatment for systemic sclerosis, dermatomyositis, cystic fibrosis, and systemic lupus erythematosus. Beyond the THC scaffold, other frameworks for synthetic cannabinoids have appeared, for example, cyclohexylphenols (e.g., CP55,940), cannabidiol derivatives (e.g., KLS-13019), cannabilactones (e.g., AM1714), and a variety of other heterocyclic scaffolds described elsewhere.

It is well understood that three-dimensional ligands can exhibit superior recognition for their biological targets compared to planar analogs. Furthermore, ligands built upon a central biaryl framework can be readily functionalized by numerous methods and are often metabolically stable. These features have made biaryls a common template in drug discovery campaigns. This quality is particularly relevant to cannabinoid design as many phytocannabinoids and synthetic variants are prone to aerobic and metabolic oxidation. Finally, axial chirality is unexplored with respect to cannabinoid ligands, providing potentially rich grounds for discovery and innovation. Thus, preserving three-dimensional structure when designing cannabinoid analogs will enable superior recognition of biological targets by these molecules when compared to their planar counterparts.

Despite advances in research directed to phytocannabinoid-based analog development, there is still a scarcity of shelf-stable cannabinoids and of accessible, systematic synthetic methods to prepare these compounds, which may serve as valuable research tools as well as leads for cannabinoid-based drug discovery. These needs and other needs are satisfied by the present disclosure.

### SUMMARY

In accordance with the purpose(s) of the present disclosure, as embodied and broadly described herein, the disclosure, in one aspect, relates to axially chiral cannabinoid analogs and methods of making the same. In one aspect, several tetracyclic scaffolds can be prepared from O-propargyl vinyl coumarins in good yields. In a further aspect, these tetracyclic scaffolds can be treated with a reductant to form the axially chiral cannabinoid analogs. In another aspect, the axially chiral cannabinoid analogs are shelf stable and maintain a three-dimensional structure during storage, enabling superior recognition of biological targets such as cannabinoid receptors. Also disclosed herein are prochiral cannabinoid analogs that can be synthesized from axially chiral cannabinoid analogs.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1A shows the major phytocannabinoids (THC, CBN, and CBD). FIG. 1B shows representative synthetic cannabinoids. FIG. 1C shows past efforts and this work: axCannabinoids as bioisosteric variants with improved physical and biological properties.

FIGS. 2A-2C show axCBN retrosynthesis (FIG. 2A), forward synthesis (FIG. 2B), and representative synthetic shortcomings (FIG. 2C).

FIG. 3 is a reaction coordinate diagram showing whether the innate [3,3] of the disclosed system's reactivity can be overturned in favor of dearomative [4+2] cycloaddition.

FIG. 4 shows cannabinoid receptor (human CB₁ and CB₂) affinity for axCBNs.

FIG. 5 shows axCBN-3 exhibits 2 distinct affinities at hCB1R (pKiHi=9.4, pKiLo=7.3), unlike CBN (pKi=6.2), suggesting high affinity binding to the active conformation. Data are mean±SEM of N=3-4 experiments performed in triplicate.

FIGS. 6A-6D show results from automated docking. axCBN-3 docked within hCB1R (FIG. 6A) and hCB2R (FIG. 6B), rac-axCBD-2 docked within hCB1R (FIG. 6C) and hCB2R (FIG. 6D). For docking scores, please see the Examples.

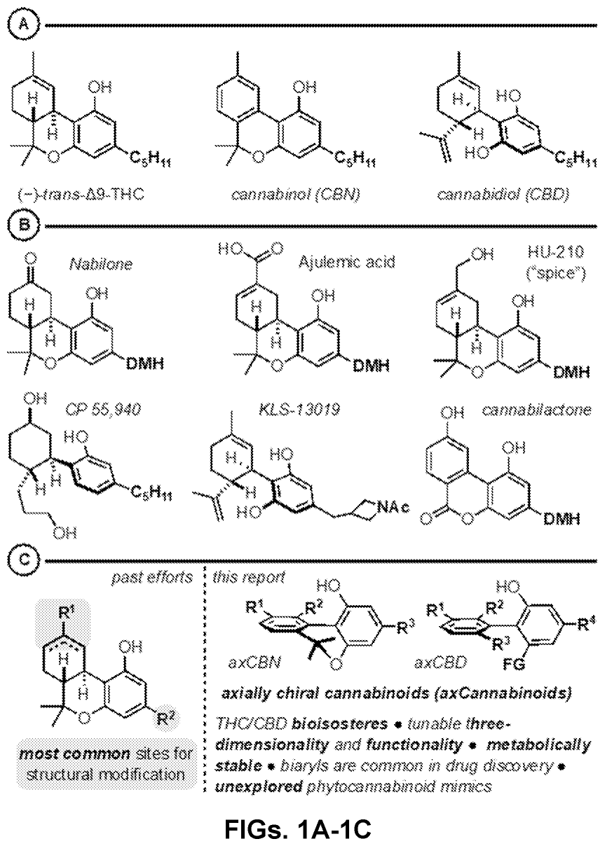

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Disclosed herein are methods for synthesizing axially chiral cannabinoids as well as axially chiral cannabinoids produced by the disclosed methods. In one aspect, the three dimensional structures of the axially chiral cannabinoids can be visually represented in the following manner:

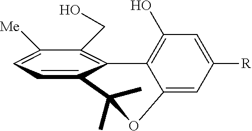

wherein it is understood that additional substituent patterns are possible depending on the starting materials for the reaction. It is further to be understood that two-dimensional representations presented elsewhere herein of axially chiral cannabinoid structures adopt the conformation pictured above.

Without wishing to be bound by theory, a thermal [4+2] cycloaddition may be impeded by a more kinetically and thermodynamically favorable propargyl Claisen rearrangement, yielding pyranocoumarins instead of the desired axially chiral cannabinoids. In one aspect, a Rh(I)-catalyzed cycloisomerization kinetically favors the desired [4+2] reactivity over the undesired [3,3] reactivity. In a further aspect, Rh-catalyzed cycloaddition can additionally be used to prepare several tetracyclic scaffolds in modest to good yields, enabling the synthesis of novel axially chiral cannabinoids.

In one aspect, disclosed herein is a synthetic platform able to convert propargyl electrophiles, crotonic acid derivatives, and 2,6-dihydroxybenzaldehyde derivatives into axially chiral cannabinoids in a convergent and concise manner.

Also disclosed are axially-chiral cannabinoids made by the disclosed method. In one aspect, the axially-chiral cannabinoids may be effective against cannabinoid receptors CB1 and CB2, as well as other targets in the central nervous system (CNS).

### Method for Synthesizing Axially Chiral Cannabinoids

In one aspect, disclosed herein is a method for synthesizing an axially chiral cannabinoid analog, the method includes at least the steps of:
(a) admixing an O-propargyl vinyl coumarin and a catalyst to form a tetracyclic scaffold compound;
(b) treating the tetracyclic scaffold compound with a reductant to form the axially chiral cannabinoid analog.

In one aspect, step (a) can occur in a first solvent and step (b) can occur in a second solvent. In another aspect, the O-propargyl vinyl coumarin has the structure:

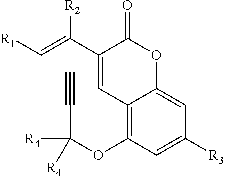

wherein R₁ is selected from hydrogen, halogen, cyano, amino, hydroxyl, optionally substituted alkyl, optionally substituted alkenyl, optionally substituted alkynyl, optionally substituted —(C0-12 alkyl)-aryl, optionally substituted —(C0-C12 alkyl)-heteroaryl, optionally substituted —(C0-C12 alkyl)-cycloalkyl, and optionally substituted —(C0-C12 alkyl)-heterocycloalkyl;
wherein R₂ is selected from C1-C12 alkyl ester, optionally substituted C1-C12 alkyl, or silyl ether;
wherein R₃ is selected from hydrogen, halo, cyano, amino, hydroxyl, optionally substituted C1-C12 alkyl, optionally substituted alkenyl, optionally substituted C1-C12 alkynyl, optionally substituted —(C0-C12 alkyl)-aryl, optionally substituted —(C0-C12 alkyl)-heteroaryl, optionally substituted —(C0-C12 alkyl)-cycloalkyl, or optionally substituted —(C0-C12 alkyl)-heterocycloalkyl; and
where in R₄ is selected from hydrogen, optionally substituted, C1-C6 alkyl, optionally substituted C3-C8 cycloalkyl, or optionally substituted C3-C8 heterocycloalkyl.

In still another aspect, the tetracyclic scaffold compound has the structure:

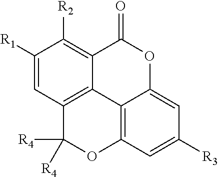

wherein R₁ is selected from hydrogen, halogen, cyano, amino, hydroxyl, optionally substituted alkyl, optionally substituted alkenyl, optionally substituted alkynyl, optionally substituted —(C0-12 alkyl)-aryl, optionally substituted —(C0-C12 alkyl)-heteroaryl, optionally substituted —(C0-C12 alkyl)-cycloalkyl, and optionally substituted —(C0-C12 alkyl)-heterocycloalkyl;
wherein R₂ is selected from C1-C12 alkyl ester, optionally substituted C1-C12 alkyl, or silyl ether;
wherein R₃ is selected from hydrogen, halo, cyano, amino, hydroxyl, optionally substituted C1-C12 alkyl, optionally substituted alkenyl, optionally substituted C1-C12 alkynyl, optionally substituted —(C0-C12 alkyl)-aryl, optionally substituted —(C0-C12 alkyl)-heteroaryl, optionally substituted —(C0-C12 alkyl)-cycloalkyl, or optionally substituted —(C0-C12 alkyl)-heterocycloalkyl; and
where in R₄ is selected from hydrogen, optionally substituted, C1-C6 alkyl, optionally substituted C3-C8 cycloalkyl, or optionally substituted C3-C8 heterocycloalkyl.

In another aspect, in either the O-propargyl vinyl coumarin or the tetracyclic scaffold, R₁ can be H or ethyl. In a further aspect, R₂ can be CO₂Et, OTBS, or methyl. In a still further aspect, R₃ can be C₅H₁₁, H, or 1,1-dimethylheptyl (DMH). In yet another aspect, R₄ can be H, methyl, or cyclohexyl.

In another aspect, the catalyst can be a rhodium catalyst such as, for example, Rh(PPh₃)₃Cl, Rh[(nbd)Cl]₂, or any combination thereof. In an aspect, Rh[(nbd)Cl]₂ is sometimes also referred to as bicyclo[2.2.1]hepta-2,5-diene-rhodium(I) chloride dimer or 2,5-norbornadiene-rhodium(I) chloride dimer. In one aspect, the catalyst can be present in an amount of about 10 mol % relative to the O-propargyl vinyl coumarin. In yet another aspect, the first solvent can be or include 2,2,2-trifluoroethanol (TFE).

In another aspect, the method further includes admixing an additive with the O-propargyl vinyl coumarin and the catalyst. In some aspects, the additive can be a silver salt such as, for example, Ag(OTf), AgSbF₆, AgBF₄, or any combination thereof. In one aspect, the additive can be present in an amount of about 10 mol % relative to the O-propargyl vinyl coumarin.

In still another aspect, step (a) can be conducted at a temperature of from about room temperature to about 120° C., or at about 20, 25, 30, 35, 40, 45, 50, 55, 60, 65, 70, 75, 80, 85, 90, 95, 100, 105, 110, 115, or about 120° C., or a combination of any of the foregoing values, or a range encompassing any of the foregoing values. In another aspect, step (a) can be carried out for about 18 hours.

In any of these aspects, the reductant can be LiAlH₄. In one aspect, the second solvent can be or include tetrahydrofuran (THF).

### Axially Chiral Cannabinoids Synthesized by the Disclosed Method

Also disclosed herein are axially chiral cannabinoid analogs synthesized by the disclosed methods and derivatives and variants thereof. In one aspect, the axially chiral cannabinoid analog or derivative or variant thereof has the structure:

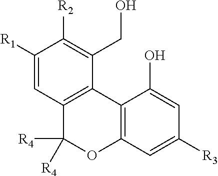

wherein R₁ is selected from hydrogen, halogen, cyano, amino, hydroxyl, optionally substituted alkyl, optionally substituted alkenyl, optionally substituted alkynyl, optionally substituted —(C0-12 alkyl)-aryl, optionally substituted —(C0-C12 alkyl)-heteroaryl, optionally substituted —(C0-C12 alkyl)-cycloalkyl, and optionally substituted —(C0-C12 alkyl)-heterocycloalkyl;
wherein R₂ is selected from C1-C12 alkyl ester, optionally substituted C1-C12 alkyl, or silyl ether;
wherein R₃ is selected from hydrogen, halo, cyano, amino, hydroxyl, optionally substituted C1-C12 alkyl, optionally substituted alkenyl, optionally substituted C1-C12 alkynyl, optionally substituted —(C0-C12 alkyl)-aryl, optionally substituted —(C0-C12 alkyl)-heteroaryl, optionally substituted —(C0-C12 alkyl)-cycloalkyl, or optionally substituted —(C0-C12 alkyl)-heterocycloalkyl; and
where in R₄ is selected from hydrogen, optionally substituted, C1-C6 alkyl, optionally substituted C3-C8 cycloalkyl, or optionally substituted C3-C8 heterocycloalkyl.

In another aspect, in either the axially chiral cannabinoid analog, R₁ can be H or ethyl. In a further aspect, R₂ can be CO₂Et, OTBS, or methyl. In a still further aspect, R₃ can be C₅H₁₁, H, or 1,1-dimethylheptyl (DMH). In yet another aspect, R₄ can be H, methyl, or cyclohexyl.

In one aspect, the axially chiral cannabinoid analog has one of the following structures, or any combination thereof:

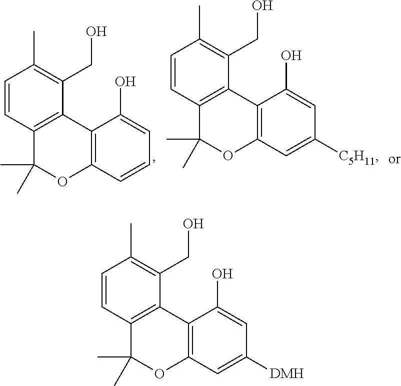

In another aspect, disclosed herein is an axially chiral cannabinoid analog having the structure:

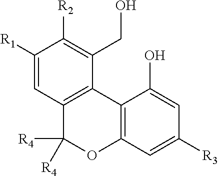

wherein R₁ is selected from hydrogen, halogen, cyano, amino, hydroxyl, optionally substituted alkyl, optionally substituted alkenyl, optionally substituted alkynyl, optionally substituted —(C0-12 alkyl)-aryl, optionally substituted —(C0-C12 alkyl)-heteroaryl, optionally substituted —(C0-C12 alkyl)-cycloalkyl, and optionally substituted —(C0-C12 alkyl)-heterocycloalkyl;
wherein R₂ is selected from C1-C12 alkyl ester or silyl ether;
wherein R₃ is selected from hydrogen, halo, cyano, amino, hydroxyl, optionally substituted C1-C12 alkyl, optionally substituted alkenyl, optionally substituted C1-C12 alkynyl, optionally substituted —(C0-C12 alkyl)-aryl, optionally substituted —(C0-C12 alkyl)-heteroaryl, optionally substituted —(C0-C12 alkyl)-cycloalkyl, or optionally substituted —(C0-C12 alkyl)-heterocycloalkyl; and
wherein each R₄ is independently selected from hydrogen, optionally substituted C1-C6 alkyl, optionally substituted C3-C8 cycloalkyl, or optionally substituted C3-C8 heterocycloalkyl.

In still another aspect, disclosed herein is an axially chiral cannabinoid analog having the structure:

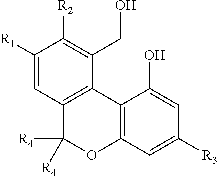

wherein R₁ is selected from hydrogen, halogen, cyano, amino, hydroxyl, optionally substituted alkyl, optionally substituted alkenyl, optionally substituted alkynyl, optionally substituted —(C0-12 alkyl)-aryl, optionally substituted —(C0-C12 alkyl)-heteroaryl, optionally substituted —(C0-C12 alkyl)-cycloalkyl, and optionally substituted —(C0-C12 alkyl)-heterocycloalkyl;
wherein R₂ is selected from C1-C12 alkyl ester, optionally substituted C1-C12 alkyl, or silyl ether;
wherein R₃ is selected from hydrogen, halo, cyano, amino, hydroxyl, optionally substituted C1-C12 alkyl, optionally substituted alkenyl, optionally substituted C1-C12 alkynyl, optionally substituted —(C0-C12 alkyl)-aryl, optionally substituted —(C0-C12 alkyl)-heteroaryl, optionally substituted —(C0-C12 alkyl)-cycloalkyl, or optionally substituted —(C0-C12 alkyl)-heterocycloalkyl; and
wherein each R₄ is independently selected from optionally substituted C3-C8 cycloalkyl or optionally substituted C3-C8 heterocycloalkyl.

### Prochiral Cannabinoid Analogs and Methods of Synthesizing the Same

Also disclosed herein is a method for synthesizing a prochiral cannabinoid analog, the method including at least the step of contacting an axially chiral cannabinoid analog with a reducing agent or a base. In one aspect, the reducing agent can be sodium ethanethiolate or the base can be lithium bis(trimethylsilyl)amide.

In one aspect, the axially chiral cannabinoid analog has a structure

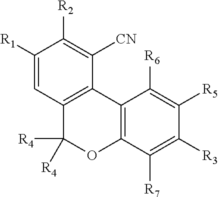

wherein R₁ is selected from hydrogen, halogen, cyano, amino, hydroxyl, optionally substituted alkyl, optionally substituted alkenyl, optionally substituted alkynyl, optionally substituted —(C0-12 alkyl)-aryl, optionally substituted —(C0-C12 alkyl)-heteroaryl, optionally substituted —(C0-C12 alkyl)-cycloalkyl, and optionally substituted —(C0-C12 alkyl)-heterocycloalkyl;
wherein R₂ is selected from C1-C12 alkyl ester, optionally substituted C1-C12 alkyl, or silyl ether;
wherein R₃, R₅, R₆, and R₇ are independently selected from hydrogen, halo, cyano, amino, hydroxyl, optionally substituted C1-C12 alkyl, optionally substituted alkenyl, optionally substituted C1-C12 alkynyl, optionally substituted —(C0-C12 alkyl)-aryl, optionally substituted —(C0-C12 alkyl)-heteroaryl, optionally substituted —(C0-C12 alkyl)-cycloalkyl, or optionally substituted —(C0-C12 alkyl)-heterocycloalkyl; and
wherein each R₄ is independently selected from optionally substituted C3-C8 cycloalkyl or optionally substituted C3-C8 heterocycloalkyl.

Also disclosed are prochiral cannabinoid analogs produced by the disclosed methods. In one aspect, the prochiral cannabinoid analog can have the structure:

The prochiral cannabinoid analog of claim 30, having a structure:

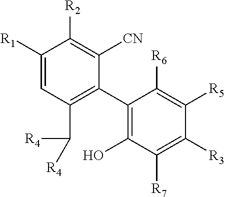

wherein R₁ is selected from hydrogen, halogen, cyano, amino, hydroxyl, optionally substituted alkyl, optionally substituted alkenyl, optionally substituted alkynyl, optionally substituted —(C0-12 alkyl)-aryl, optionally substituted —(C0-C12 alkyl)-heteroaryl, optionally substituted —(C0-C12 alkyl)-cycloalkyl, and optionally substituted —(C0-C12 alkyl)-heterocycloalkyl;
wherein R₂ is selected from C1-C12 alkyl ester, optionally substituted C1-C12 alkyl, or silyl ether;
wherein R₃, R₅, R₆, and R₇ are independently selected from hydrogen, halo, cyano, amino, hydroxyl, optionally substituted C1-C12 alkyl, optionally substituted alkenyl, optionally substituted C1-C12 alkynyl, optionally substituted —(C0-C12 alkyl)-aryl, optionally substituted —(C0-C12 alkyl)-heteroaryl, optionally substituted —(C0-C12 alkyl)-cycloalkyl, or optionally substituted —(C0-C12 alkyl)-heterocycloalkyl; and
wherein each R₄ is independently selected from optionally substituted C3-C8 cycloalkyl or optionally substituted C3-C8 heterocycloalkyl.

Further disclosed are prochiral cannabinoid analogs and/or synthetic derivatives thereof, such as, for example,

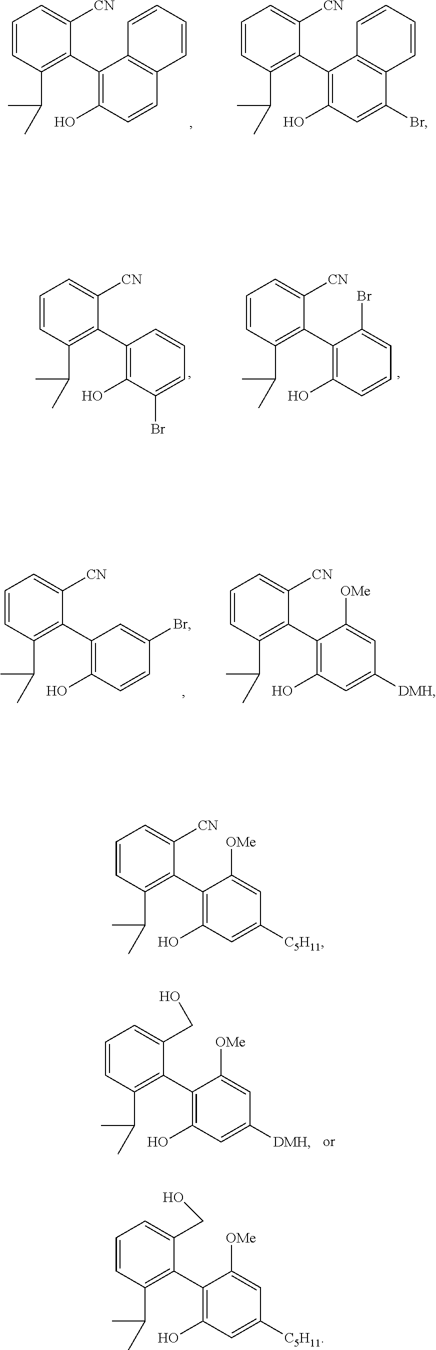

In another aspect, standard organic chemistry techniques can be employed to produce the synthetic derivatives of the prochiral cannabinoid analogs from prochiral cannabinoid analogs.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a ligand,” “a catalyst,” or “an analog,” includes, but is not limited to, mixtures or combinations of two or more such ligands, catalysts, or analogs, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

“Axially chiral” as used herein refers to a molecule that does not possess a chiral center but rather an axis of chirality. In one aspect, when a molecule possesses an axis of chirality, substituents are positioned in a spatial arrangement such that the molecule is not superimposable on its mirror image. In one aspect, the present disclosure provides for axially chiral cannabinoid analogs and a method of synthesis of the same.

“Cannabinoids” refers to a class of plant secondary metabolites that interact with a cannabinoid receptor (for example, CB1 or CB2), including, but not limited to, Δ⁹-tetrahydrocannabinol, cannabidiol, and related compounds. As used herein, a “cannabinoid analog” is a synthetic or semi-synthetic compound that interacts with a known cannabinoid receptor, a putative cannabinoid receptor (e.g., certain G protein-coupled receptors such as, for example, GPR55), or both. In some aspects, cannabinoid analogs may share structural features or substituents with known cannabinoids.

In some aspects, a reagent or additive or other reaction component may be present in a “catalytic amount” in the methods disclosed herein. As used herein, “catalytic amount” refers to an amount that is substantially less than a stoichiometric amount of any reactants or products. In one aspect, a substance present in a catalytic amount can be regenerated and reused multiple times during a reaction, and a stoichiometric amount is therefore not needed. In another aspect, a substance present in a catalytic amount is not exhausted during a reaction. In a further aspect, a catalytic amount can be less than 10% of a stoichiometric amount of a reactant or product, or less than 9, 8, 7, 6, 5, 4, 3, 2, 1, 0.5, 0.25, or less than about 0.1% of a stoichiometric amount of a reactant or product, or a combination of any of the foregoing values, or a range encompassing any of the foregoing values.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “alkanediyl” as used herein, refers to a divalent saturated aliphatic group, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂— (methylene), —CH₂CH₂—, —CH₂C(CH₃)₂CH₂—, and —CH₂CH₂CH₂— are non-limiting examples of alkanediyl groups.

The terms “alkoxy” and “alkoxyl” as used herein to refer to an alkyl or cycloalkyl group bonded through an ether linkage; that is, an “alkoxy” group can be defined as —OA′ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl to two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) and —N(-alkyl)₂, where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ or -(A¹O(O)C-A²-OC(O))ₐ, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen” or “halide,” as used herein can be used interchangeably and refer to F, Cl, Br, or I.

The terms “pseudohalide,” “pseudohalogen” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxy” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula —SiAA²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” . . . “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄Rº; —(CH₂)₀₋₄ORº; —O(CH₂)₀₋₄Rº, —O—(CH₂)₀₋₄C(O)ORº; —(CH₂)₀₋₄CH(ORº)₂; —(CH₂)₀₋₄SRº; —(CH₂)₀₋₄Ph, which may be substituted with Rº; —(CH₂)₀₋₄O(CH₂)₀₋₁Ph which may be substituted with Rº; —CH═CHPh, which may be substituted with Rº; —(CH₂)₀₋₄O(CH₂)₀₋₁-pyridyl which may be substituted with Rº; —NO₂; —CN; —N₃; —(CH₂)₀₋₄N(Rº)₂; —(CH₂)₀₋₄N(Rº)C(O)Rº; —N(Rº)C(S)Rº; —(CH₂)₀₋₄N(Rº)C(O)NRº₂; —N(Rº)C(S)NRº₂; —(CH₂)₀₋₄N(Rº)C(O)ORº; —N(Rº)N(Rº)C(O)Rº; —N(Rº)N(Rº)C(O)NRº₂; —N(Rº)N(Rº)C(O)ORº; —(CH₂)₀₋₄C(O)Rº; —C(S)Rº; (CH₂)₀₋₄C(O)ORº; —(CH₂)₀₋₄C(O)SRº; —(CH₂)₀₋₄C(O)OSiRº₃; —(CH₂)₀₋₄OC(O)Rº; —OC(O)(CH₂)₀₋₄SR—, SC(S)SRº; —(CH₂)₀₋₄SC(O)Rº; —(CH₂)₀₋₄C(O)NRº₂; —C(S)NRº₂; —C(S)SRº; —(CH₂)₀₋₄OC(O)NRº₂; —C(O)N(ORº)Rº; —C(O)C(O)Rº; —C(O)CH₂C(O)Rº; —C(NORº)Rº; —(CH₂)₀₋₄SSRº; (CH₂)₀₋₄S(O)₂Rº; —(CH₂)₀₋₄S(O)₂ORº; —(CH₂)₀₋₄OS(O)₂Rº; —S(O)₂NRº₂; —(CH₂)₀₋₄S(O)Rº; —N(Rº)S(O)₂NRº₂; —N(Rº)S(O)₂Rº; —N(ORº)Rº; —C(NH)NRº₂; —P(O)₂Rº; —P(O)Rº₂; —OP(O)Rº₂; —OP(O)(ORº)₂; SiRº₃; —(C₁₋₄ straight or branched alkylene)O—N(Rº)₂; or —(C₁₋₄ straight or branched alkylene)C(O)O—N(Rº)₂, wherein each Rº may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of Rº, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on Rº (or the ring formed by taking two independent occurrences of Rº together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{≡}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of Rº include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{≡}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

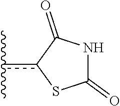

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

“Inorganic radicals,” as the term is defined and used herein, contain no carbon atoms and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, sulfur, fluorine, and chlorine, such as ²H, ³H, ¹³C, ¹⁴C ¹⁵N, ¹⁸O, ¹⁷O, ³⁵S, ¹⁸F, and ³⁶Cl, respectively. Compounds further comprise prodrugs thereof and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### Aspects

The present disclosure can be described in accordance with the following numbered aspects, which should not be confused with the claims.

Aspect 1. A method for synthesizing an axially chiral cannabinoid analog, the method comprising:
(a) admixing an O-propargyl vinyl coumarin and a catalyst to form a tetracyclic scaffold compound;
(b) treating the tetracyclic scaffold compound with a reductant to form the axially chiral cannabinoid analog.

Aspect 2. The method of aspect 1, wherein the O-propargyl vinyl coumarin has the structure:

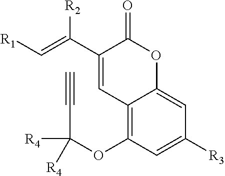

wherein R₁ is selected from hydrogen, halogen, cyano, amino, hydroxyl, optionally substituted alkyl, optionally substituted alkenyl, optionally substituted alkynyl, optionally substituted —(C0-12 alkyl)-aryl, optionally substituted —(C0-C12 alkyl)-heteroaryl, optionally substituted —(C0-C12 alkyl)-cycloalkyl, and optionally substituted —(C0-C12 alkyl)-heterocycloalkyl;
wherein R₂ is selected from C1-C12 alkyl ester, optionally substituted C1-C12 alkyl, or silyl ether;
wherein R₃ is selected from hydrogen, halo, cyano, amino, hydroxyl, optionally substituted C1-C12 alkyl, optionally substituted alkenyl, optionally substituted C1-C12 alkynyl, optionally substituted —(C0-C12 alkyl)-aryl, optionally substituted —(C0-C12 alkyl)-heteroaryl, optionally substituted —(C0-C12 alkyl)-cycloalkyl, or optionally substituted —(C0-C12 alkyl)-heterocycloalkyl; and
where in R₄ is selected from hydrogen, optionally substituted C1-C6 alkyl, optionally substituted C3-C8 cycloalkyl, or optionally substituted C3-C8 heterocycloalkyl.

Aspect 3. The method of aspect 1 or 2, wherein the tetracyclic scaffold compound has the structure:

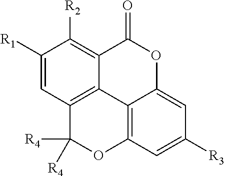

wherein R₁ is selected from hydrogen, halogen, cyano, amino, hydroxyl, optionally substituted alkyl, optionally substituted alkenyl, optionally substituted alkynyl, optionally substituted —(C0-12 alkyl)-aryl, optionally substituted —(C0-C12 alkyl)-heteroaryl, optionally substituted —(C0-C12 alkyl)-cycloalkyl, and optionally substituted —(C0-C12 alkyl)-heterocycloalkyl;
wherein R₂ is selected from C1-C12 alkyl ester, optionally substituted C1-C12 alkyl, or silyl ether;
wherein R₃ is selected from hydrogen, halo, cyano, amino, hydroxyl, optionally substituted C1-C12 alkyl, optionally substituted alkenyl, optionally substituted C1-C12 alkynyl, optionally substituted —(C0-C12 alkyl)-aryl, optionally substituted —(C0-C12 alkyl)-heteroaryl, optionally substituted —(C0-C12 alkyl)-cycloalkyl, or optionally substituted —(C0-C12 alkyl)-heterocycloalkyl; and
where in R₄ is selected from hydrogen, optionally substituted C1-C6 alkyl, optionally substituted C3-C8 cycloalkyl, or optionally substituted C3-C8 heterocycloalkyl.

Aspect 4. The method of aspect 2 or 3, wherein R₁ is H or ethyl.

Aspect 5. The method of any one of aspects 2-4, wherein R₂ is CO₂Et, OTBS, or methyl.

Aspect 6. The method of any one of aspects 2-5, wherein R₃ is C₅H₁₁, H, or 1,1-dimethylheptyl (DMH).

Aspect 7. The method of any one of aspects 2-6, wherein R₄ is H, methyl, or cyclohexyl.

Aspect 8. The method of any one of the preceding aspects, wherein the catalyst comprises a rhodium catalyst.

Aspect 9. The method of aspect 8, wherein the catalyst comprises Rh(PPh₃)₃Cl, Rh[(nbd)Cl]₂, or any combination thereof.

Aspect 10. The method of any one of the preceding aspects, wherein the catalyst is present in a catalytic amount relative to the O-propargyl vinyl coumarin.

Aspect 11. The method of any one of the preceding aspects, further comprising admixing an additive with the O-propargyl vinyl coumarin and the catalyst.

Aspect 12. The method of aspect 11, wherein the additive comprises Ag(OTf), AgSbF₆, AgBF₄, or any combination thereof.

Aspect 13. The method of aspect 11 or 12, wherein the additive is present in a catalytic amount relative to the O-propargyl vinyl coumarin.

Aspect 14. The method of any one of the preceding aspects, wherein step (a) is conducted at a temperature of from about room temperature to about 120° C.

Aspect 15. The method of any one of the preceding aspects, wherein step (a) is carried out for about 18 h.

Aspect 16. The method of any one of the preceding aspects, wherein the reductant comprises LiAlH₄.

Aspect 17. An axially chiral cannabinoid analog synthesized by the method of any one of the preceding aspects, or a derivative or variant thereof.

Aspect 18. The axially chiral cannabinoid analog or derivative or variant thereof of aspect 17, wherein the axially chiral cannabinoid analog or derivative or variant thereof comprises the structure:

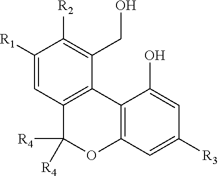

wherein R₁ is selected from hydrogen, halogen, cyano, amino, hydroxyl, optionally substituted alkyl, optionally substituted alkenyl, optionally substituted alkynyl, optionally substituted —(C0-12 alkyl)-aryl, optionally substituted —(C0-C12 alkyl)-heteroaryl, optionally substituted —(C0-C12 alkyl)-cycloalkyl, and optionally substituted —(C0-C12 alkyl)-heterocycloalkyl;
wherein R₂ is selected from C1-C12 alkyl ester, optionally substituted C1-C12 alkyl, or silyl ether;
wherein R₃ is selected from hydrogen, halo, cyano, amino, hydroxyl, optionally substituted C1-C12 alkyl, optionally substituted alkenyl, optionally substituted C1-C12 alkynyl, optionally substituted —(C0-C12 alkyl)-aryl, optionally substituted —(C0-C12 alkyl)-heteroaryl, optionally substituted —(C0-C12 alkyl)-cycloalkyl, or optionally substituted —(C0-C12 alkyl)-heterocycloalkyl; and
where in R₄ is selected from hydrogen, optionally substituted C1-C6 alkyl, optionally substituted C3-C8 cycloalkyl, or optionally substituted C3-C8 heterocycloalkyl.

Aspect 19. The axially chiral cannabinoid analog of aspect 18, wherein R₁ is H or ethyl.

Aspect 20. The axially chiral cannabinoid analog of aspect 17 or 18, wherein R₂ is CO₂Et, OTBS, or methyl.

Aspect 21. The axially chiral cannabinoid analog of any one of aspects 17-20, wherein R₃ is C₅H₁₁, H, or 1,1-dimethylheptyl (DMH).

Aspect 22. The axially chiral cannabinoid analog of any one of aspects 17-21, wherein R₄ is H, methyl, or cyclohexyl.

Aspect 23. The axially chiral cannabinoid analog of any one of aspects 17-22, wherein the axially chiral cannabinoid analog has the structure

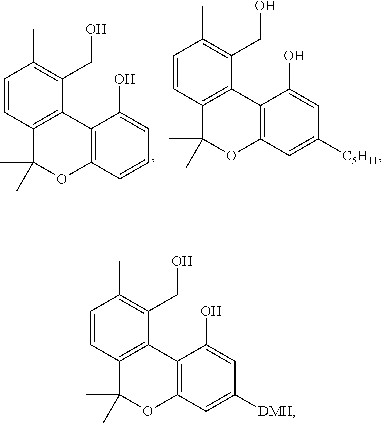

or any combination thereof.

Aspect 24. The axially chiral cannabinoid analog of aspect 17, wherein the axially chiral cannabinoid analog has an affinity for cannabinoid receptor 1 (CB1), cannabinoid receptor 2 (CB2), or both CB1 and CB2 of less than 1 nM.

Aspect 25. The axially chiral cannabinoid analog of aspect 17, wherein the axially chiral cannabinoid analog has an selectivity for CB2 at least 4.5-fold higher than a non-chiral cannabinoid with otherwise identical substituents.

Aspect 26. An axially chiral cannabinoid analog comprising the structure:

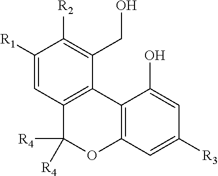

wherein R₁ is selected from hydrogen, halogen, cyano, amino, hydroxyl, optionally substituted alkyl, optionally substituted alkenyl, optionally substituted alkynyl, optionally substituted —(C0-12 alkyl)-aryl, optionally substituted —(C0-C12 alkyl)-heteroaryl, optionally substituted —(C0-C12 alkyl)-cycloalkyl, and optionally substituted —(C0-C12 alkyl)-heterocycloalkyl;
wherein R₂ is selected from C1-C12 alkyl ester or silyl ether;
wherein R₃ is selected from hydrogen, halo, cyano, amino, hydroxyl, optionally substituted C1-C12 alkyl, optionally substituted alkenyl, optionally substituted C1-C12 alkynyl, optionally substituted —(C0-C12 alkyl)-aryl, optionally substituted —(C0-C12 alkyl)-heteroaryl, optionally substituted —(C0-C12 alkyl)-cycloalkyl, or optionally substituted —(C0-C12 alkyl)-heterocycloalkyl; and
wherein each R₄ is independently selected from hydrogen, optionally substituted C1-C6 alkyl, optionally substituted C3-C8 cycloalkyl, or optionally substituted C3-C8 heterocycloalkyl.

Aspect 27. An axially chiral cannabinoid analog comprising the structure:

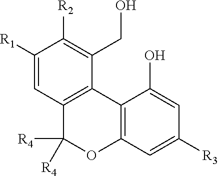

wherein R₁ is selected from hydrogen, halogen, cyano, amino, hydroxyl, optionally substituted alkyl, optionally substituted alkenyl, optionally substituted alkynyl, optionally substituted —(C0-12 alkyl)-aryl, optionally substituted —(C0-C12 alkyl)-heteroaryl, optionally substituted —(C0-C12 alkyl)-cycloalkyl, and optionally substituted —(C0-C12 alkyl)-heterocycloalkyl;
wherein R₂ is selected from C1-C12 alkyl ester, optionally substituted C1-C12 alkyl, or silyl ether;
wherein R₃ is selected from hydrogen, halo, cyano, amino, hydroxyl, optionally substituted C1-C12 alkyl, optionally substituted alkenyl, optionally substituted C1-C12 alkynyl, optionally substituted —(C0-C12 alkyl)-aryl, optionally substituted —(C0-C12 alkyl)-heteroaryl, optionally substituted —(C0-C12 alkyl)-cycloalkyl, or optionally substituted —(C0-C12 alkyl)-heterocycloalkyl; and
wherein each R₄ is independently selected from optionally substituted C3-C8 cycloalkyl or optionally substituted C3-C8 heterocycloalkyl.

Aspect 28. A method for synthesizing a prochiral cannabinoid analog, the method comprising contacting an axially chiral cannabinoid analog with a reducing agent or a base

Aspect 29. The method of aspect 28, wherein the axially chiral cannabinoid analog has a structure:

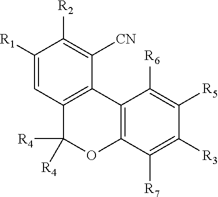

wherein R₁ is selected from hydrogen, halogen, cyano, amino, hydroxyl, optionally substituted alkyl, optionally substituted alkenyl, optionally substituted alkynyl, optionally substituted —(C0-12 alkyl)-aryl, optionally substituted —(C0-C12 alkyl)-heteroaryl, optionally substituted —(C0-C12 alkyl)-cycloalkyl, and optionally substituted —(C0-C12 alkyl)-heterocycloalkyl;
wherein R₂ is selected from C1-C12 alkyl ester, optionally substituted C1-C12 alkyl, or silyl ether;
wherein R₃, R₅, R₆, and R₇ are independently selected from hydrogen, halo, cyano, amino, hydroxyl, optionally substituted C1-C12 alkyl, optionally substituted alkenyl, optionally substituted C1-C12 alkynyl, optionally substituted —(C0-C12 alkyl)-aryl, optionally substituted —(C0-C12 alkyl)-heteroaryl, optionally substituted —(C0-C12 alkyl)-cycloalkyl, or optionally substituted —(C0-C12 alkyl)-heterocycloalkyl; and
wherein each R₄ is independently selected from optionally substituted C3-C8 cycloalkyl or optionally substituted C3-C8 heterocycloalkyl.

Aspect 30. The method of aspect 28, wherein the reducing agent comprises sodium ethanethiolate.

Aspect 31. The method of aspect 28, wherein the base comprises lithium bis(trimethylsilyl)amide.

Aspect 32. A prochiral cannabinoid analog produced by the method of any one of aspects 28-31.

Aspect 33. The prochiral cannabinoid analog of aspect 32, having a structure:

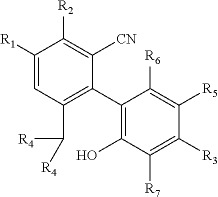

wherein R₁ is selected from hydrogen, halogen, cyano, amino, hydroxyl, optionally substituted alkyl, optionally substituted alkenyl, optionally substituted alkynyl, optionally substituted —(C0-12 alkyl)-aryl, optionally substituted —(C0-C12 alkyl)-heteroaryl, optionally substituted —(C0-C12 alkyl)-cycloalkyl, and optionally substituted —(C0-C12 alkyl)-heterocycloalkyl;
wherein R₂ is selected from C1-C12 alkyl ester, optionally substituted C1-C12 alkyl, or silyl ether;
wherein R₃, R₅, R₆, and R₇ are independently selected from hydrogen, halo, cyano, amino, hydroxyl, optionally substituted C1-C12 alkyl, optionally substituted alkenyl, optionally substituted C1-C12 alkynyl, optionally substituted —(C0-C12 alkyl)-aryl, optionally substituted —(C0-C12 alkyl)-heteroaryl, optionally substituted —(C0-C12 alkyl)-cycloalkyl, or optionally substituted —(C0-C12 alkyl)-heterocycloalkyl; and
wherein each R₄ is independently selected from optionally substituted C3-C8 cycloalkyl or optionally substituted C3-C8 heterocycloalkyl.

Aspect 34. The prochiral cannabinoid analog of aspect 33, wherein R₁ is H or ethyl.

Aspect 35. The prochiral cannabinoid analog of aspect 33, wherein R₂ is CO₂Et, OTBS, or methyl.

Aspect 36. The prochiral cannabinoid analog of aspect 33, wherein R₃ is C₅H₁₁, H, or 1,1-dimethylheptyl (DMH).

Aspect 37. The prochiral cannabinoid analog of aspect 33, wherein each R₄ is H, methyl, or cyclohexyl.

Aspect 38. The prochiral cannabinoid analog of aspect 33, wherein R₅ is halogen or hydrogen.

Aspect 39. The prochiral cannabinoid analog of aspect 33, wherein R₆ is halogen or hydrogen.

Aspect 40. The prochiral cannabinoid analog of aspect 33, wherein R₆ is halogen or hydrogen.

Aspect 41. The prochiral cannabinoid analog of aspect 33, wherein R₅ and R₆ together form a C5-C7 aromatic or heteroaromatic ring.

Aspect 42. The prochiral cannabinoid analog of aspect 33 or a synthetic derivative thereof, wherein the prochiral cannabinoid analog or synthetic derivative is selected from:

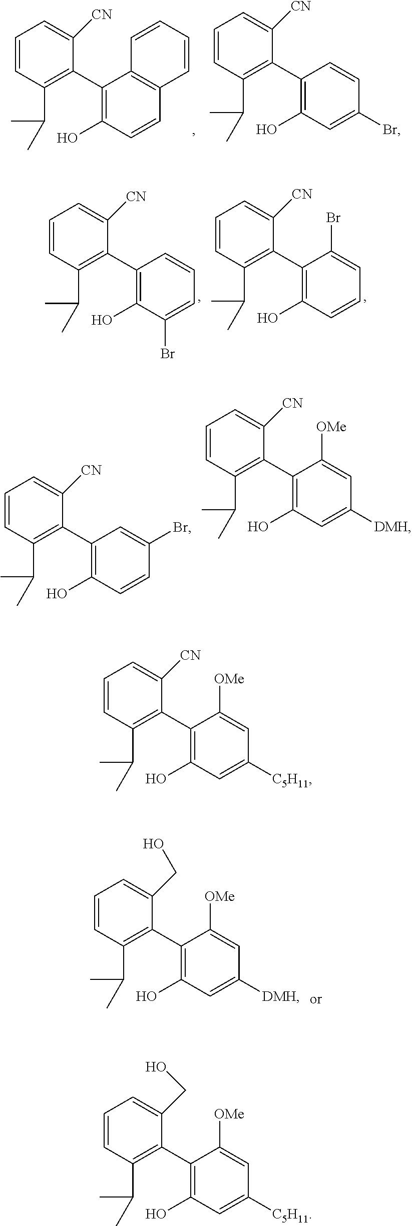

Aspect 43. The prochiral cannabinoid analog of aspect 33 or a synthetic derivative thereof, wherein the prochiral cannabinoid analog or synthetic derivative has an affinity for cannabinoid receptor 1 (CB1), cannabinoid receptor 2 (CB2), or both CB1 and CB2 of less than 1 nM.

Aspect 44. The prochiral cannabinoid analog of aspect 33 or a synthetic derivative thereof, wherein the prochiral cannabinoid analog or synthetic derivative has an selectivity for CB2 at least 4.5-fold higher than a non-chiral cannabinoid with otherwise identical substituents.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Synthetic Methods Toward Axially Chiral Cannabinols (axCBNs)

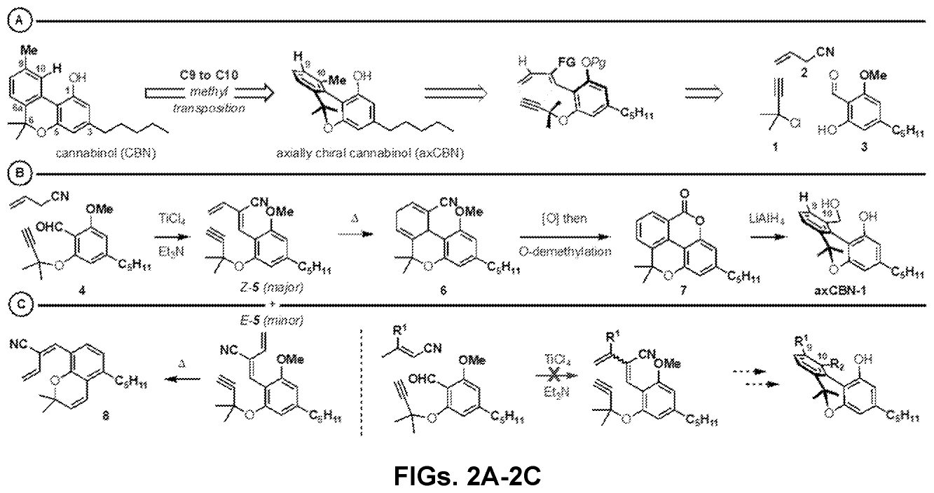

A scalable, first-generation synthesis of “parent” axially chiral cannabinol (axCBN) was previously reported, the C9-to-C10 methyl-transposed isomer of cannabinol (CBN) (FIG. 2A). By design, this transposition results in significant topological changes to the cannabinoid architecture: the ground state biaryl dihedral angle increases from 19° in CBN to 38° in axCBN. CBNs are relatively planar with little barrier to inversion, whereas axCBNs have significant three-dimensionality with barriers to atropisomersim ranging from 14-17 kcal/mol (class 1 atropisomerisim). Retrosynthetically, envisaged access to axCBN was envisaged via an intramolecular Diels-Alder approach to biaryls (DAB). This revealed dimethylpropargyl chloride 1, allylcyanide 2, and the olivetol derivative 3 as potential starting materials. A successful route to axCBN and other C10-substituted analogs was achieved through a key biaryllactone intermediate 7 (FIG. 2B). This advanced scaffold was pre-pared via Cu-catalyzed dimethylpropargylation between 1 and 3 (yielding 4), TiCl₄/Et₃N-promoted condensation between 4 and allylcyanide 2 (yielding an inseparable mixture of E5 and Z5), intramolecular Diels-Alder cycloaddition (yielding 6), DDQ oxidation, and demethylative Pinner reaction (yielding 7). axCBN-1 was prepared via LiAlH₄ reduction (FIG. 2B) and “parent” axCBN was prepared in two additional steps (FIG. 2C).

While this initial synthetic strategy provided ample amounts of parent axCBN and axCBN-1 over a reasonably efficient synthetic sequence (6-8 steps from 1, 2, and 3), it is not without shortcomings. Synthetic challenges include a non-selective vinylogous aldol condensation that produces an inseparable mixture of E5 and Z5, and only the Z isomer reacts as desired in the subsequent step. As shown in FIG. 2C the E5 isomer undergoes a propargyl Claisen rearrangement to benzochromene 8. More significantly, the goal of preparing diverse analogs with variable C9-substitution was thwarted as the key TiCl₄/Et₃N-promoted condensation between substituted crotonitriles (R≠H) was unsuccessful.

The issues encountered during the initial studies (FIG. 2C) prompted us to explore an alternative protocol for accessing axCBNs bearing both C9 and C10 substitution, as it was hypothesized that the most active axCBN analogs would have substituents at both positions. Simple transposition of the C9 methyl group to the C10 position generates a “methyl void” on the parent scaffold, and methyl groups are known to have significant impact on drug properties (the “magic methyl effect”). Consequently, deletion of the C9 methyl substituent may negatively impact the affinity and efficacy at cannabinoid receptors. Thus, the aim was to develop a synthetic route capable of facilitating diverse C9 and C10 substitution as well as variation at other positions. In this regard, access to axCBN analogs from 12 was envisioned by a sequential intramolecular aldol condensation/Diels-Alder cycloaddition yielding the advanced tetracyclic intermediate 14 (Scheme 1A). Upon cyclohexadiene oxidation and biaryl lactone ring opening, axCBN analogs would be unveiled. It was postulated that the key scaffold 12 could be prepared simply from the requisite olivetol-aldehyde 9, 3,3-dimethylacrylic acid 10, and dimethylpropargyl chloride 1, with known literature procedures for preparing aryl dimethylpropargyl ethers and divinylcoumarins serving as inspiration.

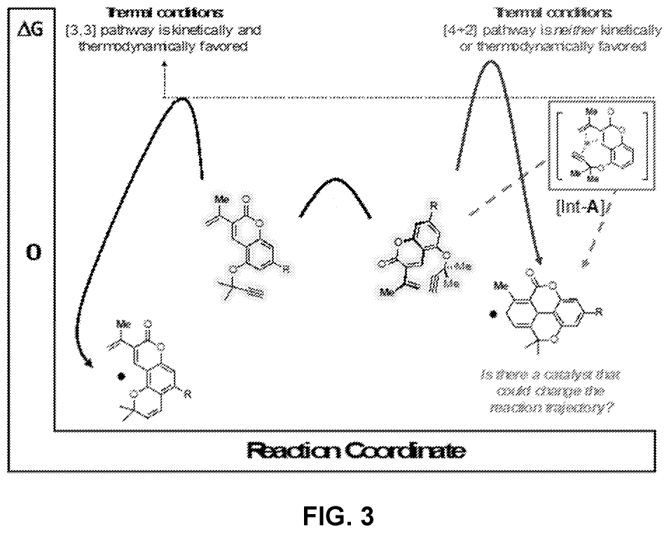

At the outset of these studies, model Diels-Alder precursors 13a-13c were successfully prepared by the proposed Cu-catalyzed dimethylpropargyl ether synthesis, phenol acylation with 3,3-dimethylacrylic acid 10, and intramolecular vinylogous aldol condensation. At this point, it was realized that the desired thermal [4+2]transformation would be more challenging than initially anticipated: under thermal conditions, these substrates exclusively react via propargyl Claisen rearrangement to yield pyranocou-marins. It became apparent that a critical Curtin-Hammett kinetics challenge exists in which the desired product 14 is neither thermodynamically nor kinetically favored over the propargyl Claisen rearrangement (FIG. 3). Recall from FIGS. 2A-2C that the Z-cyano-1,3-diene underwent favorable [4+2] cycloaddition over propargyl Claisen rearrangement. While 13 has the correct 1,3-diene geometry, the kinetics and thermodynamics of the desired [4+2] cycloaddition are less favorable due to the aromaticity of the coumarin (which must be broken during the Diels-Alder reaction). Thus, to achieve the desired transformation, reversal of the innate Curtin-Hammett controlled reactivity is necessary. Toward this goal, a transition-metal catalyst was envisioned to template the diene and dienophile (via intermediate-A (Int-A); FIG. 3), resulting in an altered kinetic profile and mechanism favoring formation of the coumarin-dearomatized [4+2] product (FIGS. 2A-2C). While there are many examples of metal catalyzed [4+2]cycloisomerization, vinylcoumarins as dienes, dearomatization, and Curtin-Hammett kinetics challenges are novel to this research area.

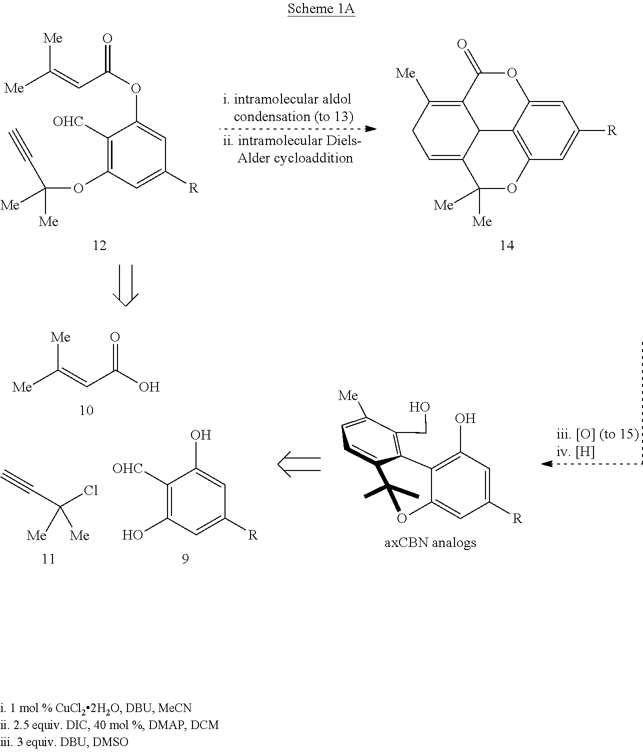

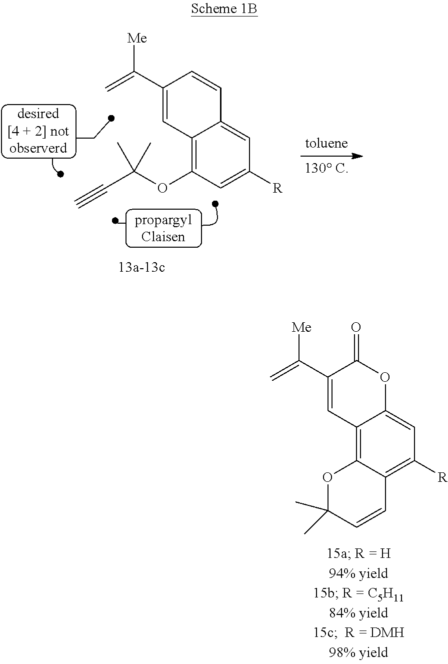

### Example 2: Synthetic Methods Toward Axially Chiral Cannabinoids (axCBNs)

To achieve the desired [4+2] reactivity, rhodium(I) catalysis was examined (Scheme 2A and Table 1).

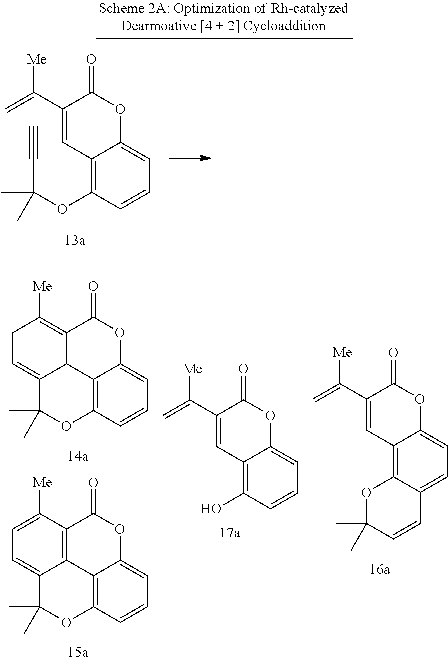

TABLE 1
Optimization of Rh-Catalyzed [4 + 2] Cycloisomerization Reaction

| | Catalyst | Additive | Solvent | Temperature | Conversion | 14a | 15b | 17a | 16a |
|---|---|---|---|---|---|---|---|---|---|
| Entry | (10 mol %) | (10 mol %) | (0.5M) | (° C.) | (%) | (% yield) | (% yield) | (% yield) | (% yield) |
| 1 | Rh(PPh₃)₃Cl | — | TFE | 55 | 100 | 10 | 9 | 3 | 29 |
| 2 | Rh(PPh₃)₃Cl | Ag(OTf) | TFE | 55 | 100 | 32 | 20 | — | — |
| 3 | Rh[(nbd)Cl]₂ | AgSbF₆ | TFE | rt | 100 | 38 | — | 23 | — |
| 4 | Rh[(nbd)Cl]₂ | Ag(OTf) | TFE | rt | 100 | 12 | 15 | 28 | — |
| 5 | Rh[(nbd)Cl]₂ | AgBF₄ | TFE | rt | 100 | 4 | 33 | — | — |
| 6 | Rh[(cod)Cl]₂ | AgSbF₆ | TFE | rt | 100 | 68 | — | 13 | — |
| 7 | Rh[(cod)Cl]₂ | Ag(OTf) | TFE | rt | 100 | 40 | 42 | — | — |
| 8 | Rh[(cod)Cl]₂ | AgBF₄ | TFE | rt | 100 | 57 | 14 | — | — |
| 9 | — | — | TFE | 55 | 10 | — | — | — | ~10 |

Using Wilkinson's catalyst, (PPh₃)₃RhCl, in trifluoroethanol (entry 1), a complex mixture of products was observed that notably contained the desired [4+2]cycloadduct 14a and its oxidation product, biaryl 15a. Also observed was the depropargylated product 17a and the propargyl Claisen rearrangement product 16a. The addition of catalytic Ag(OTf) improved the result to 32% yield 14a and 20% yield 15a (entry 2). Catalytic [Rh(NBD)Cl]₂/Ag(I) additives performed comparably to Wilkson's catalyst/Ag(OTf) (entries 3-5 vs entry 2). The best results were achieved with catalytic [Rh(COD)Cl]₂/Ag(I) salts in trifluoroethanol (entries 6-8) where combined 68%-82% yields of 14a and 15a were obtained. Notably, the reaction performed similarly well on the 1 mmol scale (Scheme 2B). As a control, the reaction was examined catalyst-free in trifluoroethanol (entry 9), confirming the essential impact of the catalyst. General substitution patterns (Schemes 2C-3B) were briefly examined, targeting the tetracyclic scaffolds 15a-15f directly via a one-pot, two-step Rh(I)-catalyzed [4+2] cycloaddition followed by in situ DDQ oxidation of the resulting 1,4-cyclohexadienes to the corresponding arenes (Scheme 2C and 2E).⁵⁶ Products 15d and 15e represent variations in the diene component. Unsubstituted (15d) and ethyl substituted (15e) dienes were reasonably tolerated. In contrast, modifications to the diene electronics resulted in little to no sign of the desired products (Scheme 2D). For example, ester substrate 13d and the silyl-enol ether diene 13e were not competent Diels-Alder substrates. With respect to the propargylic substitution on the dienophile, a cyclohexyl group was tolerated yielding 15f. However, in the absence of substitution, the transformation did not occur (Scheme 2B, 13f).

The scope studies related to the Rh(I)-catalyzed [4+2] cycloaddition suggest that a variety of C8/C10 (15e) and C9/C10 (15a and 15f) disubstituted axCBNs can be accessed. Along these lines, coumarins 13b and 13c bearing the common cannabinoid aliphatic chains (pentyl and dimethylheptyl (DMH), respectively) on the resorcinol-portion of the scaffold were prepared. Gratifyingly, the Rh(I)-catalyzed [4+2] cycloaddition/oxidation sequence yielded the desired pyrano-biaryllactones 15b and 15c. LiAlH₄ reduction furnished the targeted axCBN analogs, axCBN-2 and axCBN-3.

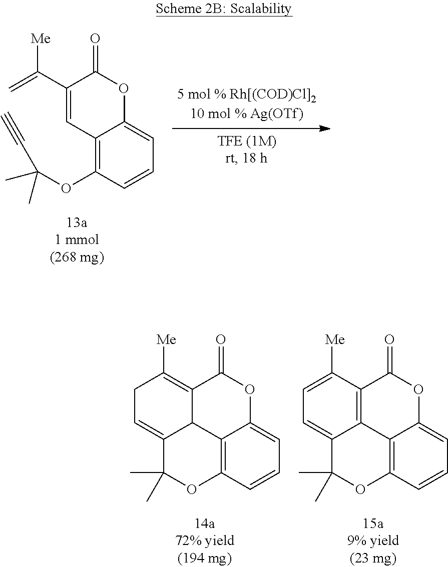

As a control, the reaction catalyst-free in trifluoroethanol (entry 9) was examined, confirming the impact of the catalyst on the transformation.

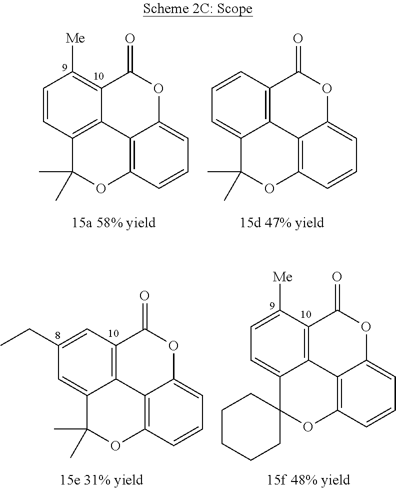

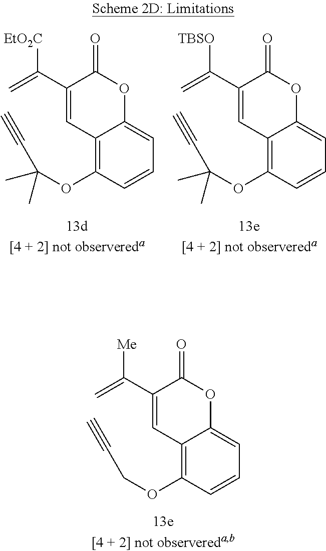

Under Rh-catalyzed conditions, the crude reaction mixture indicated depropargylated and pyranocoumarin products were present (a). Under thermal conditions (130° C., tol), less than 5% conversion was observed (b).

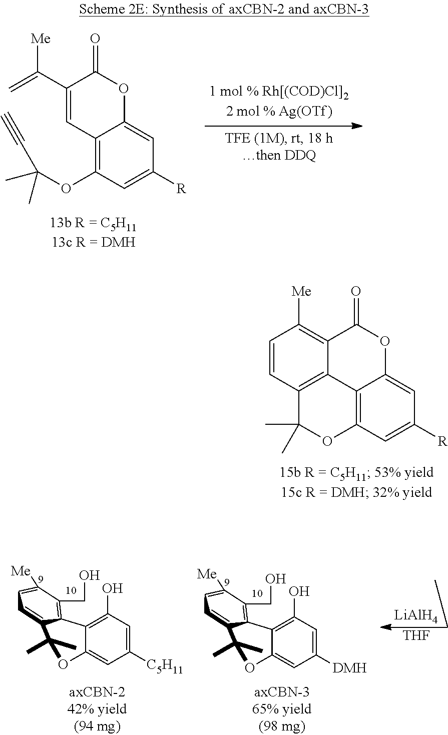

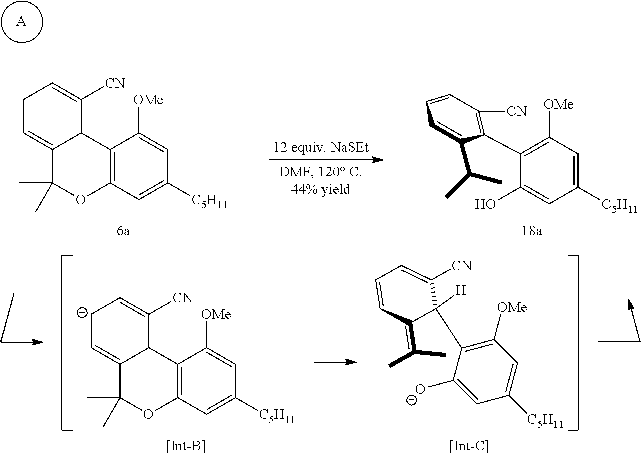

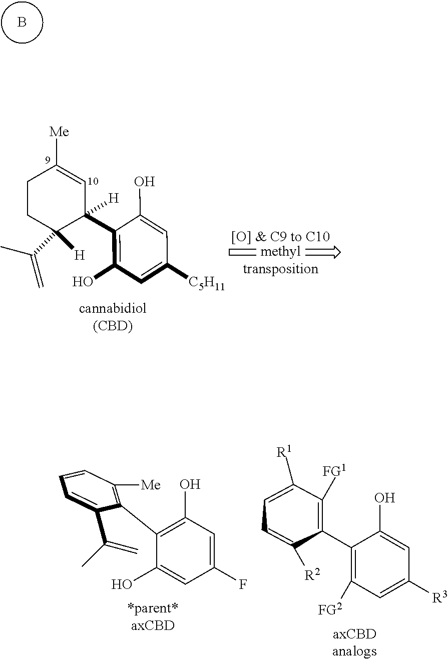

### Schemes 3A-3B

Scheme 3A shows observation of an E1_{cb} elimination reaction yielding a biaryl reminiscent of parent axCBD. Scheme 3B3 shows cannabidiol (CBD) and axially chiral cannabidiol (axCBD).

### Example 3: Synthetic Methods Toward Axially Chiral Cannabidiols (axCBDs)

During the studies related to the first-generation route to axCBNs (FIGS. 2A-2B), specifically, the attempted demethylation to free the phenol on 6, a transformation was encountered that converted the Diels-Alder adduct 6 into the biaryl 18 with concomitant pyran ring cleavage. It was surmised that this transformation occurred by an “E1_{cb} aromatization.” In this process, the nitrile group directs deprotonation yielding int-B, which is poised for pyran ring-cleavage to phenoxide int-C. In-situ or upon acidic work up, int-C undergoes a thermodynamically favorable isomerization from the nonaromatic isotoluene to the biaryl product 18. This was an interesting outcome as it resulted in an axially chiral biaryl by a unique method, and the structure is reminiscent of the theoretical “parent” axially chiral cannabidiol (axCBD). Regarding the method, Diels-Alder reaction between dienes and alkynes to yield arenes usually relies on oxidation of the intermediate 1,4-cyclohexadiene or elimination of an endocyclic leaving group.^{59,60} Thus, this represents a unique strategy for targeting substituted and functionalized arenes. With respect to CBD, axCBD is formulated in analogy to the relationship between THC and axCBN: the cyclohexene ring of the parent natural product is formally oxidized to the arene, and the methyl group is transposed from the C9 to the C10 position, thus resulting in axially chiral cannabidiols. This term should be considered loosely as parent axCBD is prochiral rather than chiral, but it bears an orthogonal, conformationally restricted biaryl linkage and thus is three-dimensional. That said, many axCBD analogs have the potential to be axially chiral biaryls.

Intrigued by the initial result and the potential to mimic the structure of cannabidiol (CBD) with axially chiral analogs, a model substrate was designed to optimize the E1_{cb} aromatization sequence for targeting axCBDs. The key substrates (6a-6i) were prepared by the same synthetic sequence outlined in FIGS. 2A-2C: (i.) TiCl₄/Et₃N-mediated aldol condensation between allyl cyanide and the requisite O-dimethylpropargylsalicylaldehyde then (ii) intramolecular Diels-Alder cycloaddition. It was found that various bases could instigate the E1_{cb} aromatization, but LiHMDS was optimal (see the supporting information for select optimization reactions). This reaction can be performed on the gram scale, and a variety of unique o-benzonitrile-o′-phenol biaryls were prepared in good yields under the optimized protocol. Notably, halogen functional handles are tolerated at every position about the phenol (18c-18g), and an o-benzonitrile-naphthol biaryl 18h is accessible. With the goal of applying this method to the synthesis of axCBD analogs, Diels-Alder adducts 6a and 6i were accessed on the 0.5-1 gram scale; these precursors bear the aliphatic chains common to bioactive cannabinoids. Under the standard E1_{cb} aromatization conditions, the advanced axially chiral biaryl intermediates 18a and 18h were prepared in good yields (54% and 44%, respectively). Nitrile reduction to the alcohol was achieved via sequential addition of DIBALH and NaBH₄ yielding axCBD-1 (R=pentyl) and axCBD-2 (R=dimethylheptyl (DMH)).

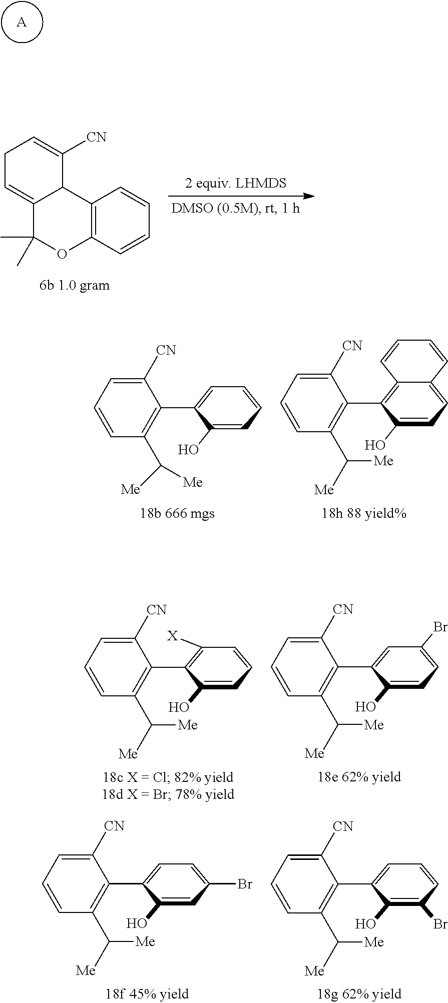

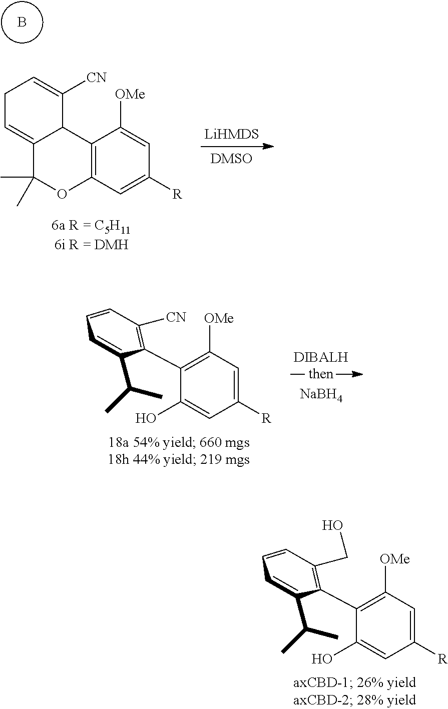

### Schemes 4A-4B3

Scheme 4A shows biaryl synthesis via E1_{cb} aromatization and Scheme 4B3 shows synthesis of axCBD-1 and axCBD-2 using E1_{cb} aromatization.

### Example 4: Atropoisomerism of Axially Chiral Cannabinoids

Axially chiral cannabinols (axCBNs) and cannabidiols (axCBDs) differ from their respective natural product counterparts, THC and CBD, by oxidation of the natural cyclohexene to a benzene ring and C9 to C10 methyl transposition. These molecules are biaryl but three-dimensional in the ground state, hence the term axially chiral cannabinoid. Using VT-NMR, the barrier to atropisomerism of axCBN-1 and its bis-acetate were determined to be 14 kcal/mol and 17 kcal/mol, respectively (Schemes 5A-5B3). Regarding axCBDs, VT-NMR experiments indicated that the biaryl linkage was conform ationally stable: no coalescence of the enantiotopic signals was observed up to 95° C. in toluene-D₈. These studies revealed that two classes of axially chiral cannabinoid have been synthesized thus far: axCBNs are class 1 atropisomers while axCBDs are class 3 atropisomers. Regarding axCBNs, they are three-dimensional in their ground state (biaryl dihedral angle=38°), but rapidly equilibrating. Thus, both enantiomeric conformers are accessible under ambient conditions.

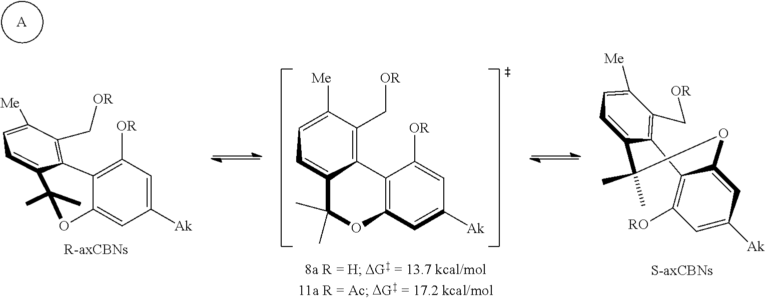

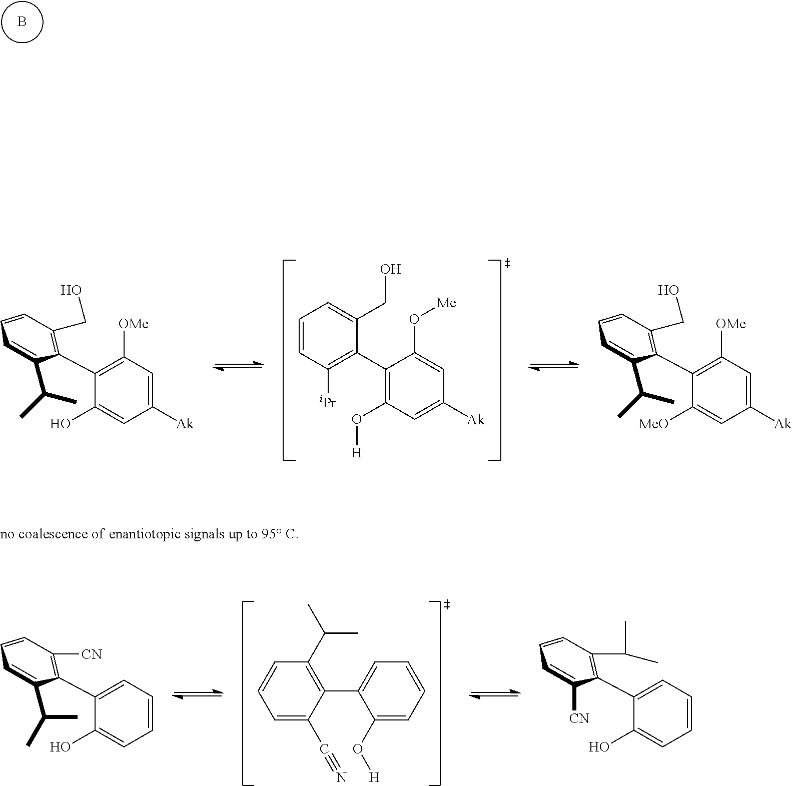

### Schemes 5A-5B

Scheme 5A shows axCBNs displaying “type 1” atropoisomerism. Scheme 5B shows axCBDs displaying “type 3” atropoisomerism.

### Example 5: Affinity of Axially Chiral Cannabinoids at Cannabinoid Receptors

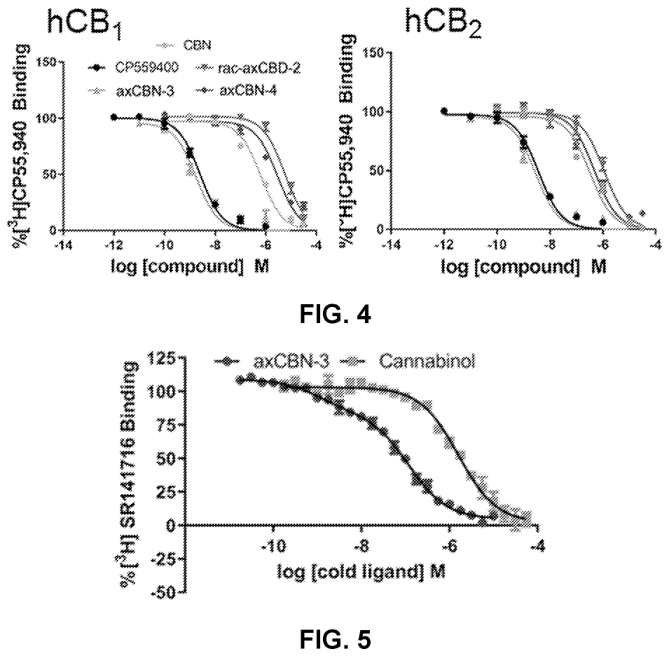

A small series of axCannabinoids has been examined for binding affinity and functional activity at human cannabinoid receptors (hCB1R and hCB2R) (Scheme 6, Tables 2 and 3, and FIGS. 4-5). From this initial series, several compounds emerged with desirable pharmacology relative to the relevant parent phytocannabinoid in terms of affinity and selectivity at human cannabinoid receptors. axCBN-3 exhibited sub-nanomolar affinity for both receptors, approximately 360-fold higher than CBN at hCB1R and 134-fold higher at hCB2R. When compared to the non-chiral dimethylheptyl derivative of CBN (CBN-DMH) reported by Rhee, et al., axCBN-3 has approximately 5-10-fold higher affinity. This suggests that the addition of the C-10 group confers additional beneficial interactions with hCB1R and hCB2R that results in higher affinity. Additionally, axCBD-2 and axCBN-4 exhibited increased selectivity for hCB2R, 4.8- and 6.2-fold respectively. Importantly, these compounds maintained functional activity as determined by stimulation of [35S]GTPγS binding, an assay of native G protein activation. axCBN-3 exhibited a 2-fold greater potency to stimulate [35S]GTPγS binding at hCB2R than at hCB1R while exhibiting increased efficacy over CBN. axCBN-4 also maintained agonist activity at hCB2R. Notably, axCBD-2 exhibited agonism at hCB2R in the TRUPATH assay of Gαiβ3γ9 protein activation, but not at hCB1R at concentrations up to 31.6 μM (FIG. 5), suggesting a potential route for development of selective CB2R agonists. This functional activity diverges from that of the parent CBD which exhibits no efficacy at either cannabinoid receptor (data not shown). Interestingly, in contrast to CBN [F(2, 64)=1.21, p=0.305], axCBN-3 [F(2, 91)=42.7, p<0.0001] exhibited distinct affinities for two binding sites following an extra sum-of-squares F test for one site vs. two site binding models (Scheme 7). The higher affinity binding may reflect selection for the active conformation, which is also supported by the higher efficacy of axCBN-3 in [35S]GTPγS binding (data not shown). Further, because axCBN-3 is a class 1 atropisomer, it exists as two enantiomeric conformers rapidly equilibrating which each may have unique affinities for different conformations and give rise to multiphasic binding curves as depicted in FIG. 4. Thus, these compounds may exhibit particularly unique pharmacology in terms of the receptor populations they could stabilize to give rise to unique signaling profiles.

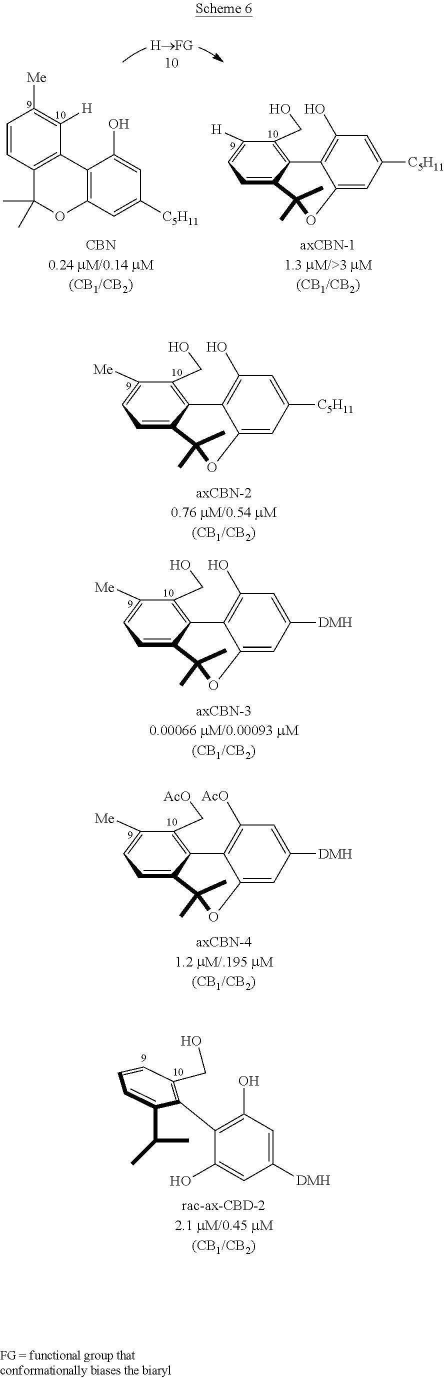

TABLE 2
Cannabinoid Receptor Affinity for axCBDs

| | hCB1R | | hCB2R | | |
|---|---|---|---|---|---|
| | pKi ± SEM | Ki (nM) | pKi ± SEM | Ki (nM) | CB1/CB2 |
| CP55,940 | 9.03 ± 0.0292 | 0.933 | 8.86 ± 0.107 | 1.38 | 0.7 |
| CBN | 6.62 ± 0.0627 | 240 | 6.86 ± 0.0370 | 138 | 1.7 |
| CBD | 5.48 ± 0.0605 | 3310 | 5.85 ± 0.0374 | 1410 | 2.3 |
| axCBD-2 | 5.67 ± 0.0821 | 2140 | 6.35 ± 0.0755 | 447 | 4.8 |
| axCBN-3 | 9.18 ± 0.0794 | 0.661 | 9.03 ± 0.141 | 0.933 | 0.7 |
| axCBN-4 | 5.92 ± 0.125 | 1200 | 6.71 ± 0.122 | 195 | 6.2 |

TABLE 3
pEC₅₀ and Eₘₐₓ of axCBDs for Human Cannabinoid Receptors^{a}

| | hCB1R | | hCB2R | |
|---|---|---|---|---|
| | pEC₅₀ | Eₘₐₓ | pEC₅₀ | Eₘₐₓ |
| CP55,940 | 9.09 ± 0.476 | 0.0913 ± 0.0173 | 9.17 ± 0.108 | 0.121 ± 0.0298 |
| axCBN-3 | 7.58 ± 0.763 | 0.0807 ± 0.0141 | 8.46 ± 0.182 | 0.120 ± 0.0296 |
| axCBN-4 | 5.15 ± 0.954 | 0.175 ± 0.0832 | 6.75 ± 0.397 | 0.120 ± 0.0294 |
| axCBD-2 | NA | NA | 7.54 ± 0.381 | 0.0839 ± 0.0176 |

^{a}Values reflect mean ± SEM of at least n = 3 experiments.
^{b}NA = no observable agonism at concentrations up to 31.6 μM.

Together, these data showcase that axCBNs and axCBDs can mimic the activity of phyto- and synthetic cannabinoids at cannabinoid receptors. These analogs occupy a unique conformational chemical space (ground-state three-dimensional structures), which impacts affinity and selectivity for biological targets (cannabinoid receptors and beyond). These molecules have the potential for improved metabolic and aerobic stability, and they represent a new platform for drug discovery and development inspired by cannabinoids.

### Example 6: Molecular Modeling of axCannabinoids at Cannabinoid Receptors

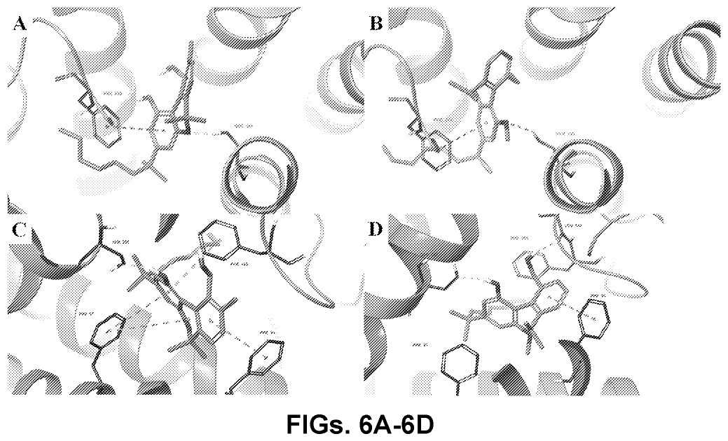

Induced-fit docking (Glide-XP, Schrödinger, Inc.) was used to predict how axCannabinoids axCBN-3 and rac-axCBD-2 engage hCB1R and hCB2R. The Glide-XP docking scoring function is an approximated binding affinity that is used to rank predicted poses of a ligand as a result of its interaction with a target: axCBN-3 had an appreciable score with hCB1R (XPgscore—13.285 kcal/mol) and hCB2R (XPgscore—13.711 kcal/mol), and rac-axCBD-2 had lower predicted affinity in hCB1R (XPgscore—12.488 kcal/mol) compared to hCB2R (XPgscore—13.117 kcal/mol). In each case, the dimethylheptyl tails of the axCannabinoids occupies the same narrow hydrophobic channel between transmembrane helix (TMH) 3, 5, and 6 as the co-crystalized ligand (FIGS. 6A-6D). Hydrophobic aromatic interactions with Phe268 (CB1R) and Phe87, Phe94 and Phe183 (CB2R) are also observed. Canonical structure-activity relationships (SAR) of classical cannabinoids indicate that a free phenol at position 1 is generally required for CB1R and CB2R binding. For axCBN-3, this group is not predicted to form beneficial interactions with CB1R, but is predicted to donate a hydrogen bond to Ser285 in CB2R; the equivalent Ser383 in CB1R does donate a hydrogen bond to the oxygen in the pyran ring. The 10-hydroxymethyl group of axCBN-3 is predicted to form an intramolecular hydrogen bond (IMHB) with the nearby 1-phenol. This may contribute to target binding by overall lowering the hydrophilicity of this region, allowing this group to occupy an otherwise hydrophobic portion of the binding site. The predicted binding pose of rac-axCBD-2 differs from that of axCBN-3 due to the larger dihedral angle connecting the two phenyl rings (ranging from −27.9° to 29.2° for axCBN-3 S# and −75.1° to −75.3° for rac-axCBD-2 S# when docked in hCB1R and hCB2R respectively). Within C1R, the 6-propyl substituent points toward Phe268, while the 10-hydroxymethyl group occupies a narrow lipophilic region below the plane of aryl ring A. Multiple steric clashes contribute to a lower Glide score, including a clash with Ser383. Within CB2R, however, the 10-hydroxymethyl group is able to donate a hydrogen bond to the backbone carbonyl of Leu182, and the phenol forms a beneficial hydrogen bond with Ser285. The presence of these additional beneficial binding interactions may explain the observation that rac-axCBN-2 is a selective CB2R agonist.

### Example 7: Conclusions and Outlook

AxCannabinoids have been conceptualized and validated as novel leads for cannabinoid-inspired drug discovery. It is hypothesized that axCannabinoids will be uniquely valuable scaffolds due to their three-dimensionality and stability imparted by the central axially chiral biaryl framework. Through the development of various de novo synthetic routes and collaborative biological analysis at cannabinoid receptors (CB₁/CB₂), a preliminary understanding of axCannabinoid structure-activity relationships has been achieved. With respect to synthesis, disclosed herein are three distinct synthetic strategies capable of producing diverse analogs bearing either a tricyclic cannabinol framework or a bicyclic scaffold inspired by cannabidiol: axially chiral cannabinols (axCBNs) or cannabidiols (axCBDs), respectively. Numerous products were obtained, including 8 analogs which were examined for biological activity, and it is speculated that analogs beyond those disclosed herein are accessible with the protocols established herein. The initial structure-activity relationship study revealed an axCannabinoid (axCBN-3) with picomolar affinity for the CB₁ and CB₂ receptors as well as other promising leads (e.g. axCBN-4 and rac-axCBD-2) that display >5-fold selectivity for the CB₂ receptor over the CB₁ receptor. The axCannabinoids described here offer new opportunities to probe the binding sites of cannabinoid receptors (C1R, CE2R) and other protein targets of phytocannabinoids.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Abu-Sawwa, R.; et al. Emerging Use of Epidiolex (Cannabidiol) in Epilepsy. J Pediatr Pharmacol Ther 2020, 25 (6), 485-499.
2. Ametovski, A. et al. Enantioselective Total Synthesis of (−)-Δ 9-Tetrahydrocannabinol via NHeterocyclic Carbene Catalysis. Org. Lett. 2019, 21 (4): 1212-1215.
3. Ashburn, B. O.; et al. Diels-Alder Approach to Biaryls (DAB): Importance of the Ortho-Nitro Moiety in the [4+2] Cycloaddition. Org Biomol Chem 2008, 6 (2), 255-257.
4. Ashburn, B. O.; et al. Diels-Alder Approach to Polysubstituted Biaryls: Rapid Entry to Tri- and Tetra-Ortho-Substituted Phosphorus-Containing Biaryls. Angewandte Chemie International Edition 2006, 45 (40), 6737-6741.
5. Ashburn, B. O.; et al. Synthesis of Tetra-Ortho-Substituted, Phosphorus-Containing and Carbonyl-Containing Biaryls Utilizing a Diels-Alder Approach. J Am Chem Soc 2007, 129 (29), 9109-9116.
6. B, O.; H, M.; V, G. Dronabinol. Pharma-Kritik 2022, 24 (8), 29-31.
7. Basilaia, M.; et al. Atropisomerism in the Pharmaceutically Relevant Realm. Acc Chem Res 2022, 55 (20).
8. Bloemendal, V. R. L. J.; et al. Synthetic Pathways to Tetrahydrocannabinol (THC): An Overview. Org Biomol Chem 2020, 18 (17), 3203-3215.
9. Bonnesen, P. V. et al. Catalysis of Diels-Alder Reactions by Low Oxidation State Transition-Metal Lewis Acids: Fact and Fiction. J. Am. Chem. Soc. 1989, 111 (16), 6070-6081.
10. Brafford May, M.; et al. Cancer Management and Research Dovepress Dronabinol for Chemotherapy-Induced Nausea and Vomiting Unresponsive to Antiemetics. Cancer Manag Res 2016, 8, 49-55.
11. Brenneman, D. E.; et al. Pharmacological Comparisons Between Cannabidiol and KLS-13019. J Mol Neurosci 2018, 66 (1), 121-134.
12. Bridgeman, M. B. et al. Medicinal Cannabis: History, Pharmacology, and Implications for the Acute Care Setting. P T 2017, 42 (3), 180-188.
13. Burstein, S. H. Ajulemic Acid: Potential Treatment for Chronic Inflammation. Pharmacol Res Perspect 2018, 6 (2).
14. Carreras, J. et al. Synthesis of (−)-Cannabimovone and Structural Reassignment of Anhydrocannabimovone through Gold(I)-Catalyzed Cycloisomerization. Angew. Chemie 2016, 128 (25), 7237-7241.
15. Davis, M. P. Oral Nabilone Capsules in the Treatment of Chemotherapy-Induced Nausea and Vomiting and Pain. Expert Opin Investig Drugs 2008, 17 (1), 85-95.
16. Dennis, D. G.; et al. Synthesis of the Cannabimovone and Cannabifuran Class of Minor Phytocannabinoids and Their AntiInflammatory Activity. Journal of Organic Chemistry 2022, 87 (9), 6075-6086.
17. Devinsky, O.; et al. Trial of Cannabidiol for Drug-Resistant Seizures in the Dravet Syndrome. New England Journal of Medicine 2017, 376 (21), 2011-2020.
18. De Vries, M. et al. Dronabinol and Chronic Pain: Importance of Mechanistic Considerations. Expert Opinion on Pharmacotherapy. 2014, 15: 1525-1534.
19. Elikottil, J. et al. The Analgesic Potential of Cannabinoids. J. Opioid Manag. 2009, 5 (6): 341-357.
20. Fürstner, A. et al. Two Manifolds for Metal-Catalyzed Intramolecular Diels-Alder Reactions of Unactivated Alkynes. Angew. Chemie Int. Ed. 2007, 46 (46), 8845-8849.
21. Gilbertson, S. R. et al. Rhodium Catalyzed Intramolecular [4+2] Cycloisomerization Reactions. Tetrahedron Lett. 1998, 39 (15), 2075-2078.
22. Gordo, J. et al. Convenient Synthesis of 3-Vinyl and 3-Styryl Coumarins. Org. Lett., 2011, 13 (19):5112-5115.
23. Jin, S.; et al. Gold(I)-Initiated Cycloisomerization/Diels-Alder/Retro-Diels-Alder Cascade Strategy to Biaryls. J Org Chem 2017, 82 (17), 9066-9074.
24. Jolly, R. S. et al. Novel Cyclization Reactions on Transition-Metal Templates. The Catalysis of Intramolecular [4+2] Cycloadditions by Low Valent Rhodium Complexes. J. Am. Chem. Soc. 1990, 112 (12), 4965-4966.
25. Kotha, S.; et al. Diversity-Oriented Approaches to Polycyclics and Bioinspired Molecules via the Diels-Alder Strategy: Green Chemistry, Synthetic Economy, and Beyond. ACS Comb Sci 2015, 17 (5), 253-302.
26. Lautens, M. et al. Transition Metal-Mediated Cycloaddition Reactions. Chem. Rev. 1996, 96 (1), 49-92.
27. Liu, Y.; et al. Synthesis of Functionalized Cannabilactones. Molecules 2020, 25 (3), 684.
28. Lovering, F. et al. Escape from Flatland: Increasing Saturation as an Approach to Improving Clinical Success. J. Med. Chem. 2009, 52 (21), 6752-6756.
29. Maiocchi, A.; et al. Stereoselective Synthetic Strategies to (−)-Cannabidiol. ChemistrySelect 2022, 7 (28), e202202400.
30. Mechoulam, R. et al. A Historical Overview of Chemical Research on Cannabinoids. Chem. Phys. Lipids 2000, 108 (1), 1-13.
31. Minami, T. et al. 3-Vinylcoumarins and 3-Vinylchromenes as Dienes. Application to the Synthesis of 3,4-Fused Coumarins and Chromenes. J. Org. Chem. 2002, 57 (1), 167-173.
32. Minuti, L.; et al. High-Pressure-Promoted Diels—Alder Approach to Biaryls: Application to the Synthesis of the Cannabinols Family. Journal of Organic Chemistry 2012, 77 (18), 7923-7931.
33. Minuti, L.; et al. High-Pressure-Promoted Diels-Alder Approach to Biaryls: Application to the Synthesis of the Cannabinols Family. J Org Chem 2012, 77 (18), 7923-7931.
34. Munjal, M.; et al. Polymeric Systems for Amorphous Δ9-Tetrahydrocannabinol Produced by a Hot-Melt Method. Part II: Effect of Oxidation Mechanisms and Chemical Interactions on Stability. J Pharm Sci 2006, 95 (11), 2473-2485.
35. Murakami, M. et al. Directed Intermolecular [4+2] Cycloaddition of Unactivated 1,3-Diene Substrates with High Regio- and Stereoselectivities. J. Am. Chem. Soc. 1997, 119 (30), 7163-7164.
36. Nalbandian, C. J. et al. The Preorganization of Atropisomers to Increase Target Selectivity. Synlett 2016, 27 (7), 977-983.
37. Navaratne, P. V. et al. Axially Chiral Cannabinols: A New Platform for Cannabinoid-Inspired Drug Discovery. ChemMedChem 2020, 15 (9), 728-732.
38. Nicolaou, K. C. et al. Total Synthesis of Artochamins F, H, I, and J through Cascade Reactions. Tetrahedron 2008, 64 (21), 4736-4757.
39. Nielsen, S. et al. The Use of Cannabis and Cannabinoids in Treating Symptoms of Multiple Sclerosis: A Systematic Review of Reviews. Current Neurology and Neuroscience Reports, 2018, 18 (8): 12pp.
40. Pearson, E. L. et al. Controlling Cis/Trans-Selectivity in Intramolecular Diels-Alder Reactions of Benzo-Tethered, Ester Linked 1,3,9-Decatrienes. Org. Biomol. Chem. 2008, 6 (3), 513-522.
41. Pearson, E. L. et al. Experimental and Computational Studies into an ATPH-Promoted Exo-Selective IMDA Reaction: A Short Total Synthesis of Δ9-THC. Chem.—A Eur. J. 2010, 16 (28), 8280-8284.
42. Pottie, I. R. et al. Synthesis of 6H-Dibenzo[b, d]Pyran-6-Ones Using the Inverse Electron Demand Diels-Alder Reaction. J. Org. Chem. 2011, 76 (21), 9015-9030.
43. Saito, A. et al. Rh(I)—Catalyzed Mild Intramolecular [4+2] Cycloaddition Reactions of Ester-Tethered Diene-Yne Compounds. Tetrahedron Lett. 2006, 47(6), 891-895.
44. Saldaña-Shumaker, S. L.; et al, Modern Approaches to the Development of Synthetic Cannabinoid Receptor Probes. Pharmacol Biochem Behav 2021, 203, 173119.
45. Salo, O. M. H. et al. Development of a 3D Model for the Human Cannabinoid CB1 Receptor. J. Med. Chem. 2004, 47 (12), 3048-3057.
46. Schafroth, M. A. et al. Synthesis of Phytocannabinoids. Prog. Chem. Org. Nat. Prod. 2017, 103, 37-59.
47. Schçnherr, H.; et al. Profound Methyl Effects in Drug Discovery and a Call for New CH Methylation Reactions. Angewandte Chemie International Edition 2013, 52 (47), 12256-12267.
48. Schlitzer, S. C. et al. A Metal-Free Aromatic Cascade for the Synthesis of Diverse Heterocycles. Org. Chem. Front. 2020, 7 (7), 913-918.
49. Schmidt, B. et al. A One-Pot Synthesis of Pyranocoumarins Through Microwave-Promoted Propargyl Claisen Rearrangement/Wittig Olefination. European J. Org. Chem. 2018, 2018 (2), 223-227.
50. Scotter, E. L. et al. The Endocannabinoid System as a Target for the Treatment of Neurodegenerative Disease. Br. J. Pharmacol. 2010, 160 (3), 480.
51. Shultz, Z. P. et al. Enantioselective Total Synthesis of Cannabinoids—A Route for Analogue Development. Org. Lett. 2018, 20 (2), 381-384.
52. Smith, D. E. et al Exploiting Atropisomerism to Increase the Target Selectivity of Kinase Inhibitors. Angew. Chemie 2015, 127 (40), 11920-11925.
53. Tai, S.; et al. Synthetic Cannabinoids: Pharmacology, Behavioral Effects, and Abuse Potential. Curr Addict Rep 2014, 1 (2), 129.
54. Tejedor, D. et al. Propargyl Claisen Rearrangement: Allene Synthesis and Beyond. Chem. Soc. Rev 2013, 42, 458-471.
55. Toenjes, S. T. et al. Atropisomerism in Medicinal Chemistry: Challenges and Opportunities. Future Med. Chem. 2018, 10 (4), 409-422.
56. US Food and Drug Administration, FDA and Cannabis: Research and Drug Approval Process, <https://www.fda.gov/newsevents/public-health-focus/fda-and-cannabis-research-and-drug-approval-process> (accessed Sep. 28, 2020).
57. Walker, D. et al. 2,3-Dichloro-5,6-Dicyanobenzoquinone and Its Reactions. Chem. Rev. 2002, 67 (2), 153-195.
58. Wender, P. A. et al. Nickel-Catalyzed Intramolecular [4+2] Dienyne Cycloadditions: An Efficient New Method for the Synthesis of Polycycles Containing Cyclohexa-1,4-Dienes, J. Am. Chem. Soc. 1989, 111 (16): 6432-6434.
59. Wender, P. A. et al. Transition Metal-Catalyzed Intramolecular [4+2] Cycloadditions: Mechanistic and Synthetic Investigations. Tetrahedron 1998, 54 (7), 1255-1275.
60. Wissel, J. et al. Low Dose Treatment with the Synthetic Cannabinoid Nabilone Significantly Reduces Spasticity-Related Pain: A Double-Blind Placebo-Controlled Cross-over Trial. J. Neurol. 2006, 253 (10), 1337-1341.
61. Wu, J. et al. Derivatives of Natural Product Agrimophol as Disruptors of Intrabacterial PH Homeostasis in Mycobacterium tuberculosis. ACS Infect. Dis., 2019, 5 (7):1087-1104.
62. Yet, L. Privileged Structures in Drug Discovery. John Wiley & Sons, Bridgewater, NJ, 2018.

1. A method for synthesizing an axially chiral cannabinoid analog, the method comprising:
(a) admixing an O-propargyl vinyl coumarin and a catalyst to form a tetracyclic scaffold compound;
(b) treating the tetracyclic scaffold compound with a reductant to form the axially chiral cannabinoid analog.

2. The method of claim 1, wherein the O-propargyl vinyl coumarin has the structure: 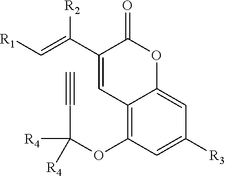
wherein R₁ is selected from hydrogen, halogen, cyano, amino, hydroxyl, optionally substituted alkyl, optionally substituted alkenyl, optionally substituted alkynyl, optionally substituted —(C0-12 alkyl)-aryl, optionally substituted —(C0-C12 alkyl)-heteroaryl, optionally substituted —(C0-C12 alkyl)-cycloalkyl, and optionally substituted —(C0-C12 alkyl)-heterocycloalkyl;
wherein R₂ is selected from C1-C12 alkyl ester, optionally substituted C1-C12 alkyl, or silyl ether;
wherein R₃ is selected from hydrogen, halo, cyano, amino, hydroxyl, optionally substituted C1-C12 alkyl, optionally substituted alkenyl, optionally substituted C1-C12 alkynyl, optionally substituted —(C0-C12 alkyl)-aryl, optionally substituted —(C0-C12 alkyl)-heteroaryl, optionally substituted —(C0-C12 alkyl)-cycloalkyl, or optionally substituted —(C0-C12 alkyl)-heterocycloalkyl; and
where in R₄ is selected from hydrogen, optionally substituted C1-C6 alkyl, optionally substituted C3-C8 cycloalkyl, or optionally substituted C3-C8 heterocycloalkyl.

3. The method of claim 1, wherein the tetracyclic scaffold compound has the structure: 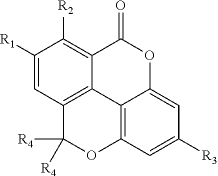
wherein R₁ is selected from hydrogen, halogen, cyano, amino, hydroxyl, optionally substituted alkyl, optionally substituted alkenyl, optionally substituted alkynyl, optionally substituted —(C0-12 alkyl)-aryl, optionally substituted —(C0-C12 alkyl)-heteroaryl, optionally substituted —(C0-C12 alkyl)-cycloalkyl, and optionally substituted —(C0-C12 alkyl)-heterocycloalkyl;
wherein R₂ is selected from C1-C12 alkyl ester, optionally substituted C1-C12 alkyl, or silyl ether;
wherein R₃ is selected from hydrogen, halo, cyano, amino, hydroxyl, optionally substituted C1-C12 alkyl, optionally substituted alkenyl, optionally substituted C1-C12 alkynyl, optionally substituted —(C0-C12 alkyl)-aryl, optionally substituted —(C0-C12 alkyl)-heteroaryl, optionally substituted —(C0-C12 alkyl)-cycloalkyl, or optionally substituted —(C0-C12 alkyl)-heterocycloalkyl; and
where in R₄ is selected from hydrogen, optionally substituted C1-C6 alkyl, optionally substituted C3-C8 cycloalkyl, or optionally substituted C3-C8 heterocycloalkyl.

4. The method of claim 2, wherein R₁ is H or ethyl.

5. The method of claim 2, wherein R₂ is CO₂Et, OTBS, or methyl.

6. The method of claim 2, wherein R₃ is C₅H₁₁, H, or 1,1-dimethylheptyl (DMH).

7. The method of claim 2, wherein R₄ is H, methyl, or cyclohexyl.

8. The method of claim 1, wherein the catalyst comprises a rhodium catalyst, wherein the rhodium catalyst comprises Rh(PPh₃)₃Cl, Rh[(nbd)Cl]₂, or any combination thereof.

9. (canceled)

10. (canceled)

11. The method of claim 1, further comprising admixing an additive with the O-propargyl vinyl coumarin and the catalyst.

12. The method of claim 11, wherein the additive comprises Ag(OTf), AgSbF₆, AgBF₄, or any combination thereof.

13. (canceled)

14. (canceled)

15. (canceled)

16. The method of claim 1, wherein the reductant comprises LiAlH₄.

17. An axially chiral cannabinoid analog synthesized by the method of claim 1, or a derivative or variant thereof.

18. The axially chiral cannabinoid analog or derivative or variant thereof of claim 17, wherein the axially chiral cannabinoid analog or derivative or variant thereof comprises the structure: 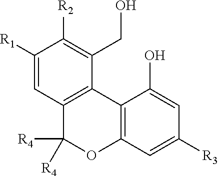
wherein R₁ is selected from hydrogen, halogen, cyano, amino, hydroxyl, optionally substituted alkyl, optionally substituted alkenyl, optionally substituted alkynyl, optionally substituted —(C0-12 alkyl)-aryl, optionally substituted —(C0-C12 alkyl)-heteroaryl, optionally substituted —(C0-C12 alkyl)-cycloalkyl, and optionally substituted —(C0-C12 alkyl)-heterocycloalkyl;
wherein R₂ is selected from C1-C12 alkyl ester, optionally substituted C1-C12 alkyl, or silyl ether;
wherein R₃ is selected from hydrogen, halo, cyano, amino, hydroxyl, optionally substituted C1-C12 alkyl, optionally substituted alkenyl, optionally substituted C1-C12 alkynyl, optionally substituted —(C0-C12 alkyl)-aryl, optionally substituted —(C0-C12 alkyl)-heteroaryl, optionally substituted —(C0-C12 alkyl)-cycloalkyl, or optionally substituted —(C0-C12 alkyl)-heterocycloalkyl; and
where in R₄ is selected from hydrogen, optionally substituted C1-C6 alkyl, optionally substituted C3-C8 cycloalkyl, or optionally substituted C3-C8 heterocycloalkyl.

19. The axially chiral cannabinoid analog of claim 18, wherein R₁ is H or ethyl.

20. The axially chiral cannabinoid analog of claim 17, wherein R₂ is CO₂Et, OTBS, or methyl.

21. The axially chiral cannabinoid analog of claim 17, wherein R₃ is C₅H₁₁, H, or 1,1-dimethylheptyl (DMH).

22. The axially chiral cannabinoid analog of claim 17, wherein R₄ is H, methyl, or cyclohexyl.

23. The axially chiral cannabinoid analog of claim 17, wherein the axially chiral cannabinoid analog has the structure 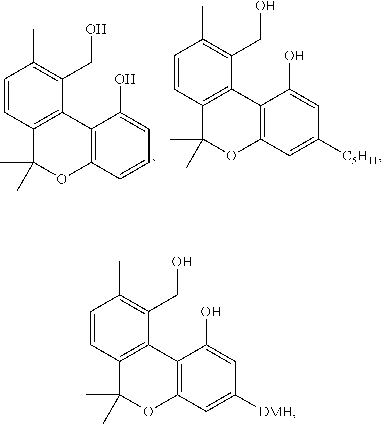
or any combination thereof.

24. The axially chiral cannabinoid analog of claim 17, wherein the axially chiral cannabinoid analog has an affinity for cannabinoid receptor 1 (CB1), cannabinoid receptor 2 (CB2), or both CB1 and CB2 of less than 1 nM.

25. The axially chiral cannabinoid analog of claim 17, wherein the axially chiral cannabinoid analog has an selectivity for CB2 at least 4.5-fold higher than a non-chiral cannabinoid with otherwise identical substituents.

26-44. (canceled)
